(19) United States
(12) Patent Application Publication
(10) Pub. No.: US 2025/0377369 A1
(43) Pub. Date: Dec. 11, 2025

(54) CONTAINER HANDLING METHOD AND AUTOMATIC MEASURING APPARATUS

(71) Applicant: SHASHIN KAGAKU CO., LTD., Kyoto-shi, Kyoto (JP)

(72) Inventors: Masahiro SHIMONOSEKI, Kyoto (JP); Masahiro HORIE, Kyoto (JP); Tatsufumi KUSUDA, Kyoto (JP)

(21) Appl. No.: 18/693,520

(22) PCT Filed: Aug. 9, 2022
(86) PCT No.: PCT/JP2022/030451

§ 371 Date: Feb. 19, 2025

(30) Foreign Application Priority Data
Oct. 15, 2021 (JP) 2021-169575

(51) Int. Cl. G01N 35/04; C12M 1/22; C12M 1/34; C12M 3/00

(52) U.S. Cl. CPC G01N 35/04; C12M 23/10; C12M 23/48; C12M 41/36; G01N 2035/0441; G01N 2035/0462

(57) ABSTRACT

In a container handling method, at a predetermined timing after having taken out the last container of a multi-stack of containers in a placement section, the last container is initially disposed in a separately predetermined location; thereafter, one container at a time is taken out from the top of a multi-stack of containers in a predetermined location and the container is disposed sequentially on the top in the separately predetermined location to form a multi-stack of containers, or one container at a time is taken out from the bottom of a multi-stack of containers in a predetermined location and the container is disposed sequentially on the bottom in the separately predetermined location to form a multi-stack of containers. By this re-stacking process, the vertical order of the containers in the multi-stack of containers returns to the original order to prevent the reversing of the order of a multi-stack of containers.

### RELATED APPLICATIONS

The present application is a National Phase of International Application Number PCT/JP2022/030451 filed Aug. 9, 2022, which claims the benefit of priority from Japanese Patent Application No. 2021-169575, filed on Oct. 15, 2021.

### TECHNICAL FIELD

The present invention relates to a container handling method and automatic measuring apparatus for handling a plurality of containers in which micro-organisms such as bacteria and fungi are cultivated.

### BACKGROUND ART

An embodiment of this type of container handling apparatus is the Petri dish handling apparatus. Such Petri dish handling apparatuses are commonly known as those built into a colony automatic measuring apparatus or a colony automatic transplanting apparatus and broadly divided into two types. As described in Patent Literatures 1 and 2, one type is an apparatus of which a Petri dish is transferred from a placement section for placing a multi-stack of Petri dishes having a plurality of stacked Petri dishes, to a measuring section or transplanting section, and then transferred from the measuring section or transplanting section to a different placement section or the original placement section. As described in Patent Literature 3, the other type is an apparatus of which a Petri dish is transferred from a placement section for placing a plurality of Petri dishes lined up in rows on a tray to a measuring section, and then transferred from the measuring section to the original placement section.

### CITATION LIST

### Patent Literature

Patent Literature 1: JP S62-267642 A

Patent Literature 2: JP 2018-121622 A

Patent Literature 3: JP 2005-261260 A

### SUMMARY OF INVENTION

### Technical Problem

In the former type of apparatus, there is an advantage in that because a plurality of containers can be stacked and placed, the placement space can be made small; hence, the placement section, and by extension the entire apparatus, can be compactly designed. However, in a multi-stack of containers formed after a measuring process or transplanting process in a separate placement section or the original placement section, the vertical order of the containers becomes reversed from that of the original order of the multi-stack of containers. Therefore, there is a disadvantage in that the next time a container is transferred from a different placement section or original placement section to a measuring section or transplanting section, and the measuring process or transplanting process is performed, the order of the measuring process or transplanting process becomes reversed.

In the latter type of apparatus, there is no disadvantage in that the order of the measuring process or transplanting process becomes reversed. However, there is a disadvantage in that because the plurality of containers must be placed by lining them up in rows on a tray, the placement space becomes large; hence the placement section, and by extension the entire apparatus, becomes large.

Therefore, with these circumstances taken into consideration, the object of the present invention is to provide a container handling method and automatic measuring apparatus which can prevent the reversing of the order of a multi-stack of containers.

### Solution to Problem

The container handling method according to the present invention, includes
transferring a container from a placement section for placing a multi-stack of containers to a measuring apparatus for measuring a sample in the container, and then transferring the container from the measuring apparatus to a predetermined location, wherein the container is taken out from a top of the multi-stack of containers in the placement section in a manner of one container at a time and the container is disposed sequentially on a top of another container in the predetermined location to form a multi-stack of containers in the predetermined location, or wherein the container is taken out from a bottom of the multi-stack of containers in the placement section in a manner of one container at a time and the container is disposed sequentially on a bottom of another container in the predetermined location to form a multi-stack of containers in the predetermined location, and
at a predetermined timing after transferring a last container of the multi-stack of containers from the placement section to the measuring apparatus and before a subsequent measuring process on the multi-stack of containers, initially disposing the last container in a separately predetermined location, and thereafter, taking out a container from a top of the multi-stack of containers in the predetermined location in a manner of one container at a time and disposing the container sequentially on a top of the last container or another container in the separately predetermined location to form a multi-stack of containers in the separately predetermined location, or taking out a container from a bottom of the multi-stack of containers in the predetermined location in a manner of one container at a time and disposing the container sequentially on a bottom of the last container or another container in the separately predetermined location to form a multi-stack of containers in the separately predetermined location.

Moreover, the automatic measuring apparatus according to the present invention is provided with
a placement section in which a multi-stack of containers can be placed,
a measuring apparatus for measuring a sample in a container,
a container transferring apparatus which can transfer the container, and
a control circuit for controlling the container transferring apparatus, wherein
controlled by the control circuit, the container transferring apparatus
transfers the container from the placement section to the measuring apparatus, and then from the measuring apparatus to a predetermined location, wherein the container is taken out from a top of the multi-stack of containers in the placement section in a manner of one container at a time and the container is disposed sequentially on a top of another container in the predetermined location to form a multi-stack of containers in the predetermined location, or wherein the container is taken out from a bottom of the multi-stack of containers in the placement section in a manner of one container at a time and the container is disposed sequentially on a bottom of another container in the predetermined location to form a multi-stack of containers in the predetermined location, and
at a predetermined timing after transferring a last container of the multi-stack of containers from the placement section to the measuring apparatus and before a subsequent measuring process on the multi-stack of containers, initially disposes the last container in a separately predetermined location, and thereafter, takes out a container from a top of the multi-stack of containers in the predetermined location in a manner of one container at a time and disposes the container sequentially on a top of the last container or another container in the separately predetermined location to form a multi-stack of containers in the separately predetermined location, or takes out a container from a bottom of the multi-stack of containers in the predetermined location in a manner of one container at a time and disposes the container sequentially on a bottom of the last container or another container in the separately predetermined location to form a multi-stack of containers in the separately predetermined location.

Here, as one mode of the automatic measuring apparatus according to the present invention, a configuration provided with
a temporary placement section in which a multi-stack of containers can be placed, wherein
the temporary placement section is set as the predetermined location, and
the placement section is set as the separately predetermined location.

Moreover, in such a case, a configuration of which
the placement section and the temporary placement section are each a container stacking apparatus comprising
   a rotary table, and
   magazines installed at equally divided angular locations around a rotary center of the rotary table, and each enabled to accommodate a multi-stack of containers, so that the multi-stack of containers can be placed on the placement section and the temporary placement section,
can be adopted.

Moreover, in such a case, a configuration in which
the placement section and the temporary placement section are disposed in areas on both sides of the measuring apparatus
can be adopted.

Furthermore, in such a case, a configuration provided with
an enclosure enclosing the placement section, the measuring apparatus, the temporary placement section and the container transferring apparatus can be adopted.

### Advantageous Effects of Invention

According to the present invention, in a multi-stack of containers formed in a predetermined location, the vertical order of the containers becomes reversed with that of the original multi-stack of containers. However, in a multi-stack of containers formed in a separately predetermined location where a re-stacking process is performed, the vertical order of the containers is the same as that of the original multi-stack of containers. Therefore, according to the present invention, the reversing of the order in a multi-stack of containers can be prevented.

### BRIEF DESCRIPTION OF DRAWINGS

FIG. 1 is a front view of an automatic measuring apparatus having a built-in colony measuring apparatus and a Petri dish handling apparatus.

FIG. 2 is a front view of an automatic measuring apparatus with an opening-and-closing cover in an opened state.

FIG. 3 is a front view of a Petri dish stacking apparatus of an automatic measuring apparatus.

FIG. 4 is a plan view of a Petri dish stacking apparatus.

FIG. 5 is a plan view of a Petri dish stacking apparatus, a colony measuring apparatus and a Petri dish handling apparatus.

FIG. 6 is a left side view of FIG. 5.

FIG. 7 is a left side view on the A-A line of FIG. 5.

FIG. 8A is an explanatory drawing of a state in which a measuring process begins.

FIG. 8B is an explanatory drawing of Operation 1 of a series of Petri dish handling operations relating to the measuring process.

FIG. 9A is an explanatory drawing of Operation 2.

FIG. 9B is an explanatory drawing of Operation 3.

FIG. 10A is an explanatory drawing of Operation 4.

FIG. 10B is an explanatory drawing of Operation 5.

FIG. 11A is an explanatory drawing of Operation 6.

FIG. 11B is an explanatory drawing of Operation 7.

FIG. 12A is an explanatory drawing of Operation 8.

FIG. 12B is an explanatory drawing of a state in which the measuring process has ended.

FIG. 13A is a flowchart of a series of Petri dish handling operations relating to the measuring process.

FIG. 13B is a flowchart of a series of Petri dish handling operations relating to a re-stacking process of the multi-stack of Petri dishes formed after the measuring process.

FIG. 14A is a conceptual diagram of multi-stack of Petri dishes before the measuring process (the original multi-stack of Petri dishes).

FIG. 14B is a conceptual diagram of multi-stack of Petri dishes formed after the measuring process.

FIG. 15 is a plan view containing a partial cross section of an automatic measuring apparatus according to another example.

FIGS. 16A and 16B are explanatory drawings showing a case in which a rotational deviation has occurred in a Petri dish.

FIG. 16C is an explanatory drawing showing a case in which a rotational deviation has occurred in an image of a plate due to the rotational deviation of the Petri dish.

FIG. 17 is a flowchart of the rotation correction process for correcting the rotational deviation of an image for the measuring process, based on the image of a plate.

FIGS. 18A to 18D are explanatory drawings of Steps 2 and 4 of FIG. 17.

FIG. 19 is a flowchart of a rotation correction process according to another example.

FIG. 20 is an explanatory drawing of the cultivation conditions equalization process for cases in which a temperature gradient occurs in the vertical direction in the cultivation environment in the automatic measuring apparatus.

FIG. 21 is an explanatory drawing of the cultivation conditions equalization process for cases in which a temperature gradient occurs in the horizontal direction in the cultivation environment in the automatic measuring apparatus.

FIG. 22 is an explanatory drawing of Another Usage Example 1.

FIG. 23 is an explanatory drawing of Another Usage Example 2.

FIG. 24 is an explanatory drawing of Another Usage Example 3 (group-based sorting process).

FIG. 25 is an explanatory drawing of Group-based Sorting Process 1.

FIG. 26 is an explanatory drawing of Group-based Sorting Process 2.

FIG. 27 is an explanatory drawing of Group-based Sorting Process 3.

FIG. 28 is an explanatory drawing of Group-based Sorting Process 4.

FIG. 29 is an explanatory drawing of Group-based Sorting Process 5.

FIG. 30 is an explanatory drawing of Another Usage Example 4 (measured results-based sorting process).

FIG. 31 is an explanatory drawing of Measured Results-based Sorting Process 1.

FIG. 32 is an explanatory drawing of Measured Results-based Sorting Process 2.

FIG. 33 is an explanatory drawing of Measured Results-based Sorting Process 3.

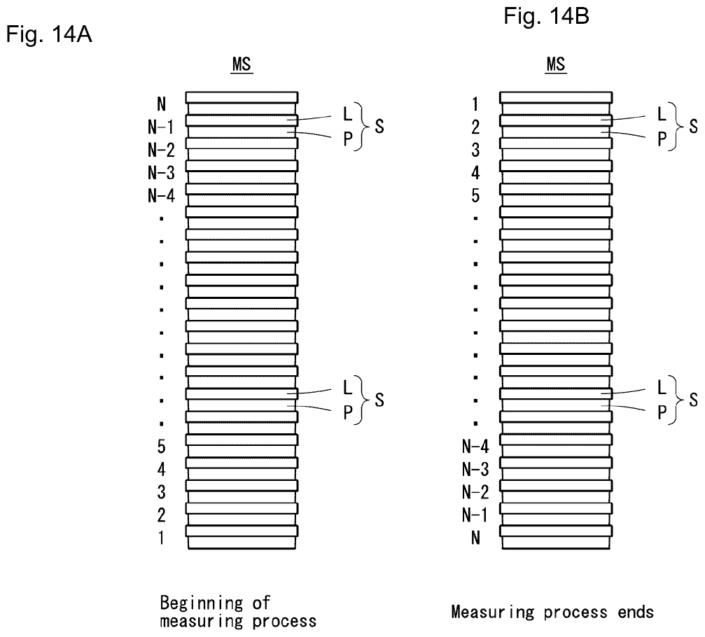

FIG. 14B is an explanatory drawing of Measured Results-based Sorting Process 4.

### DESCRIPTION OF EMBODIMENTS

### Apparatus Configuration

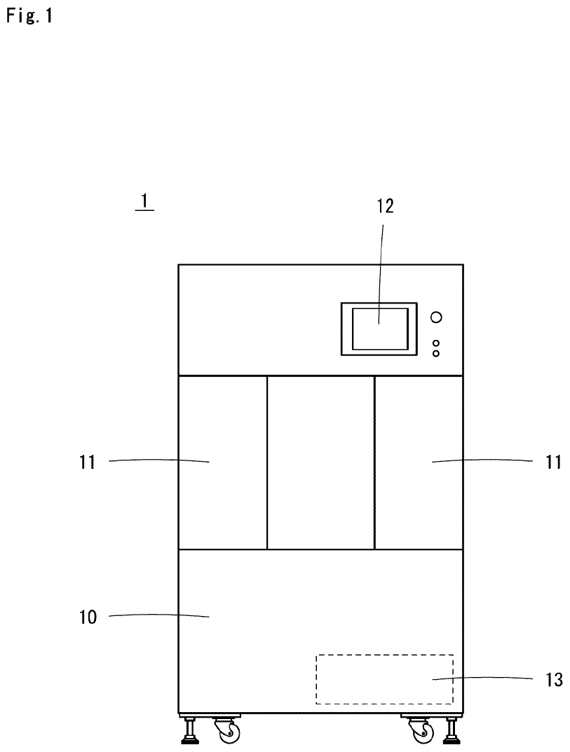

As an embodiment of a container handling apparatus according to the present invention, a Petri dish handling apparatus which is built into an automatic measuring apparatus is explained below, referring to FIGS. 1 to FIG. 14B. The case in which a Petri dish is used as a container is explained in the present embodiment; however, another container such as CompactDry™ may also be used.

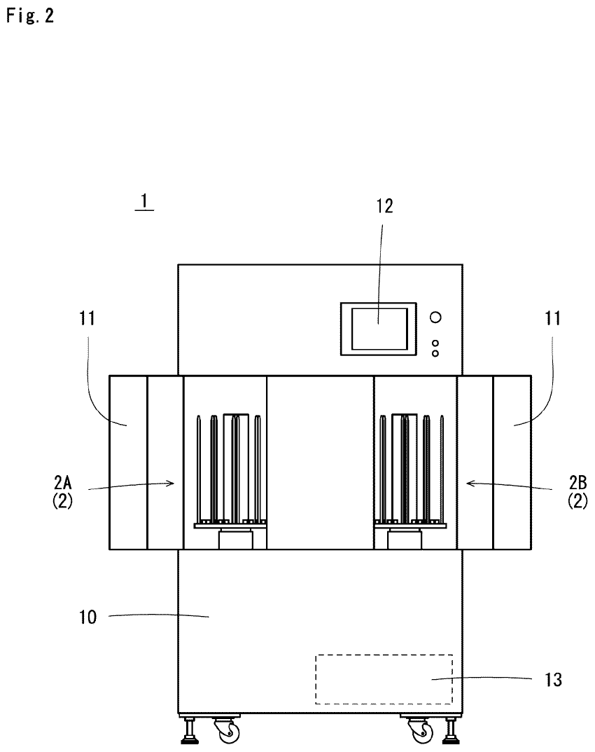

As illustrated in FIG. 1 and FIG. 2, an automatic measuring apparatus 1 is provided with an enclosure 10. The enclosure 10 is fabricated by attaching a cover as an exterior material to a frame configured by a rectangular material made from, for example, aluminum. The enclosure 10 is provided with an opening-and-closing cover 11. A pair of opening-and-closing covers 11 are installed at the left and right of the front of the enclosure 10. When opened, Petri dish stacking apparatuses (Petri dish stockers) 2 are exposed at the respective open portions, and a multi-stack of Petri dishes can be taken out and put in.

The automatic measuring apparatus 1 is provided with an operation input unit 12 and control circuit 13. The operation input unit 12 is a touch panel type which is disposed at the front of the enclosure 10, and receives all kinds of operations and information in relation to the operation of the automatic measuring apparatus 1. The control circuit 13 is configured by means of a computer such as a personal computer for example, includes a calculating unit (processor) and a recording medium (semiconductor memory, hard disk etc.), and controls the entire automatic measuring apparatus 1.

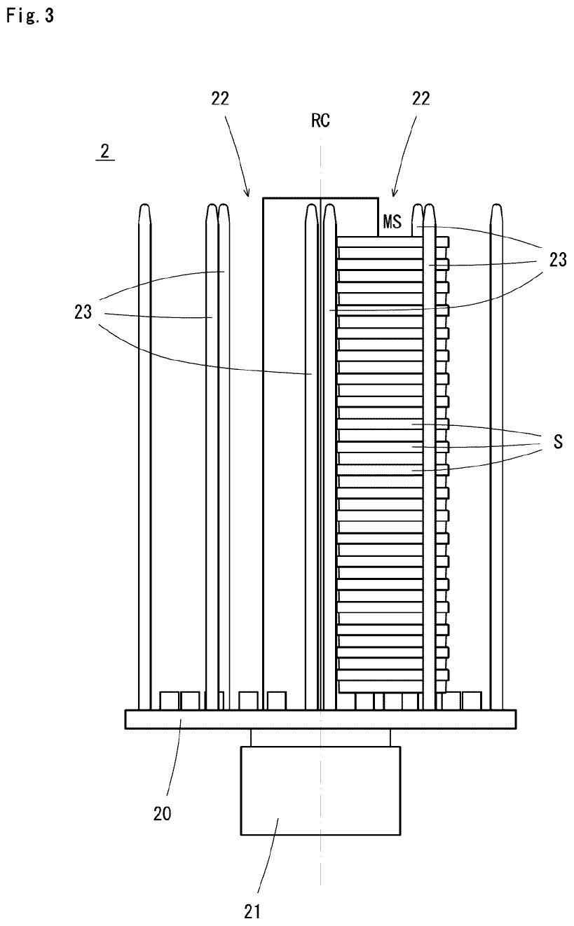

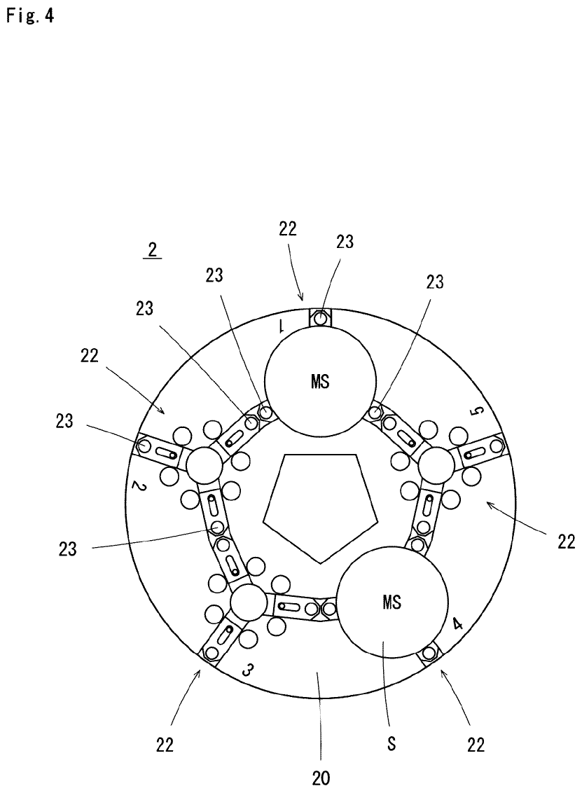

As illustrated in FIG. 3 and FIG. 4, the Petri dish stacking apparatus 2 is provided with a rotary table 20, a rotary motor 21 and a magazine 22. The rotary table 20 is a rotatable table having a vertical axis orthogonal to a horizontal surface, as a rotary center RC. The rotary motor 21 is a motor with an angle indexing function for rotating and positioning the rotary table 20 at a predetermined angular position.

A plurality of magazines 22 is installed at equally divided angular locations around the rotary center RC, where each can accommodate a multi-stack of Petri dishes MS. In the present embodiment, five magazines 22 are installed at five locations at 72-degree intervals around the rotary center RC (Magazine Numbers 1 to 5). The magazine 22 is configured by a plurality of enclosing members 23, . . . which enclose Petri dishes S. In the present embodiment, the enclosing member 23 is a bar extending upwards from the rotary table 20 at three locations at 120-degree intervals around the center of the magazine 22. The multi-stack of Petri dishes MS are accommodated so as to internally contact the plurality of enclosing members 23, . . . . The number of Petri dishes S which can be accommodated in the magazine 22 is determined by the height dimension of the enclosing member 23. In the present embodiment, the magazine 22 can accommodate 21 Petri dishes S, and the entire Petri dish stacking apparatus 2 can accommodate 105 Petri dishes S.

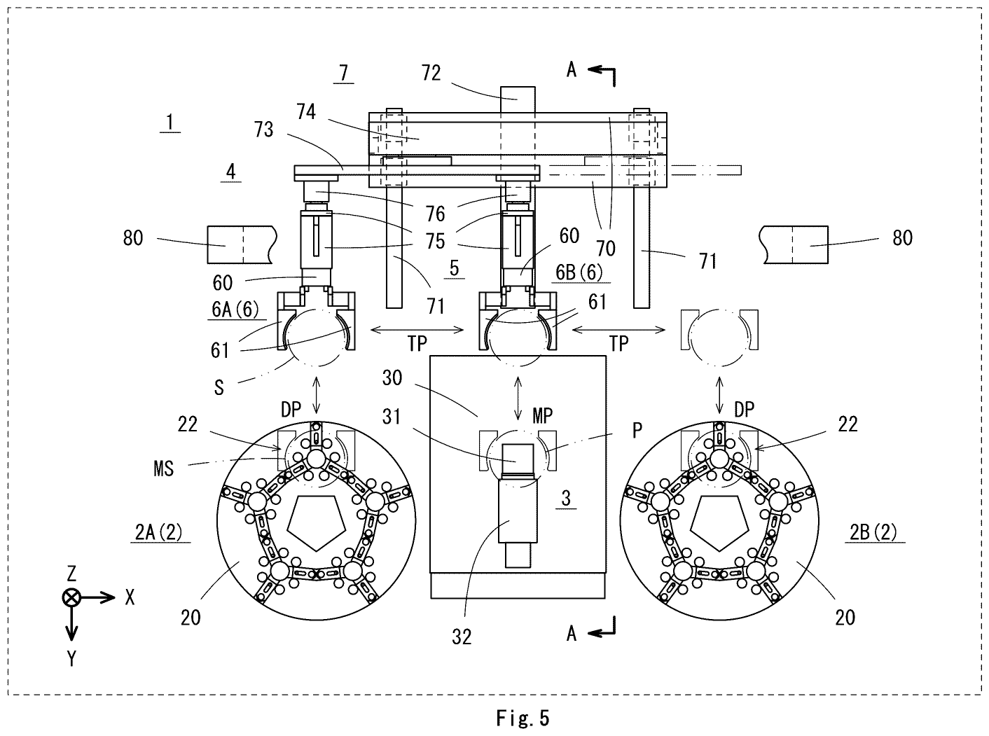

As illustrated in FIG. 5, the Petri dish stacking apparatus 2 is provided with a first Petri dish stacking apparatus 2A and a second Petri dish stacking apparatus 2B. The first Petri dish stacking apparatus 2A and second Petri dish stacking apparatus 2B are disposed with a predetermined interval in the X-direction, which is the left-right direction of the automatic measuring apparatus 1. The first Petri dish stacking apparatus 2A is used as a placement section to place and store the multi-stack of Petri dishes MS. The second Petri dish stacking apparatus 2B is used as a temporary placement section to temporarily place the Petri dishes S transferred from the first Petri dish stacking apparatus 2A. The first Petri dish stacking apparatus 2A and second Petri dish stacking apparatus 2B rotate synchronously. Namely, the first Petri dish stacking apparatus 2A and second Petri dish stacking apparatus 2B are angularly displaced synchronously so that the same magazine numbers are positioned at delivery positions DP. The positioning of the magazine 22 of a magazine number to be provided for use (the magazine 22 of the magazine number indicated by the control circuit 13) at the delivery position DP is referred to as “indexing of a magazine number”.

As illustrated in FIGS. 5 to 7, in addition to the Petri dish stacking apparatuses 2, 2 mentioned above, the automatic measuring apparatus 1 is provided with a colony measuring apparatus (colony counter) 3 and a Petri dish handling apparatus 4. The Petri dish handling apparatus 4 is provided with a Petri dish transferring apparatus (transferring robot) 5 and a Petri dish lid removing/attaching apparatus (removing/attaching robot) 8. The Petri dish transferring apparatus 5 is provided with a gripping apparatus 6 and drive apparatus 7.

The colony measuring apparatus 3 is an apparatus for measuring (counting) the number of colonies of micro-organisms cultivated in a Petri dish. The colony measuring apparatus 3 is disposed between the first Petri dish stacking apparatus 2A and the second Petri dish stacking apparatus 2B. The colony measuring apparatus 3 is provided with a stage 30 and imaging apparatus (camera) 31. A plate P of a Petri dish S which is an object to be photographed (of which a lid L was removed from the Petri dish S) is placed at a measuring position MP on the stage 30. The imaging apparatus 31 is supported by being mounted to a support frame 32 extending upwards from the stage 30, and is disposed so that an optical axis is orthogonal to the stage 30 where the center point of the measuring position MP is configured as the center of the optical axis. After subjecting an image of the plate P which was captured by the imaging apparatus 31 to a suitable imaging process, the control circuit 13, which is used as a control circuit of the colony measuring apparatus 3, executes identification processing of a colony, and then executes a colony counting process based on the result of the identification processing.

A gripping apparatus 6 is provided with a pair of jaw grippers 61, 61. The pair of jaw grippers 61, 61 holds a portion of the plate P of the Petri dish S from both sides. The pair of jaw grippers 61, 61 are mounted to a pair of action units of actuator 60 of a type in which the action units move in a swinging contacting-separating motion. The gripping apparatus 6 can thereby switch between a state in which the Petri dish S is held, and a state in which the Petri dish S is released.

The gripping apparatus 6 is provided with a first gripping apparatus 6A and a second gripping apparatus 6B. The first gripping apparatus 6A and the second gripping apparatus 6B are located at the same position in the Y-direction which is the front-back direction of the automatic measuring apparatus 1, and are disposed with a predetermined interval in the X-direction. The distance between the center line of the first gripping apparatus 6A and the center line of the second gripping apparatus 6B in the X-direction is equal to the distance between the center point of the delivery position DP of the first Petri dish stacking apparatus 2A and the center point of the measuring position MP in the X-direction, and the distance between the center point of the delivery position DP of the second Petri dish stacking apparatus 2B and the center point of the measuring position MP in the X-direction. The first Petri dish stacking apparatus 2A, the colony measuring apparatus 3 and the second Petri dish stacking apparatus 2B are disposed alongside the X-direction so that the center point of the delivery position DP of the first Petri dish stacking apparatus 2A, the center point of the measuring position MP and the center point of the delivery position DP of the second Petri dish stacking apparatus 2B are located at the same position in the Y-direction.

The drive apparatus 7 is provided with a first base 70, a second base 73 and a third base 75. The first base 70 is guided in a linear motion in the Y-direction by means of a pair of linear guides 71, 71 each disposed to the left and right along the Y-direction. Moreover, the first base 70 is mounted to an action unit of actuator 72 of a type in which the action unit moves in a linear motion, and is driven in the Y-direction. The second base 73 is mounted to an action unit of actuator 74 of a type in which the action unit moves in a linear motion, and is driven in the X-direction. The third base 75 is mounted to an action unit of actuator 76 of a type in which the action unit moves in a linear motion, and is driven in the Z-direction which is the vertical direction of the automatic measuring apparatus 1. The gripping apparatus 6 is directly, or indirectly via a member, mounted to the third base 75. The gripping apparatus 6 is thereby movable in three-dimensional space by orthogonal three axes XYZ in the movable area of actuators 72, 74, 76.

The Petri dish lid removing/attaching apparatus 8 is an apparatus of which the lid L of the Petri dish S held by the gripping apparatus 6 can be removed or attached when the gripping apparatus 6 is located on a line extending in the Y-direction from the center point of the measuring position MP, at a traversing position TP and at an upward position in the Z-direction. The Petri dish lid removing/attaching apparatus 8 is provided with a base 81 and head 83. The base 81 is mounted to an action unit of actuator 82 of a type in which the action unit moves in a linear motion, and is driven in the Z-direction. The actuator 82 is supported by being mounted to a portal type support frame 80 disposed along the X-direction. The head 83 is mounted to an action unit of actuator 85 of a type in which the action unit moves in a rotating motion, and is driven about the axis of the Z-direction. The actuator 85 is directly, or indirectly via a member, mounted to the base 81. The head 83 can thereby move in the Z-direction through the center of the Petri dish S held by the gripping apparatus 6; moreover, the head 83 can rotate and be angularly displaced about the axis of the Z-direction through the center of the Petri dish S held by the gripping apparatus 6.

The head 83 is provided with a suction nozzle 84 at the tip end side. The Petri dish lid removing/attaching apparatus 8 thereby performs suctioning to remove, or release to remove and attach, the lid L of the Petri dish S, in a state of which the head 83 has descended and has approached the Petri dish S held by the gripping apparatus 6. With rotation and angular displacement by means of actuation of the actuator 85, the head 83 can rotate and angularly displace the lid L about the axis in the Z-direction through the center of the Petri dish S held by the gripping apparatus 6 in a state in which suction was performed on the lid L.

The Petri dish lid removing/attaching apparatus 8 is provided with a reading device 86. The reading device 86 is an apparatus for reading specific information on a Petri dish such as a sample name or lot information which is recorded on an information recording medium such as a bar code, a QR code (TM) or an RF-ID tag which was assigned to the upper surface or circumferential side surface of the lid L of the Petri dish S, or the circumferential side surface of the plate P of the Petri dish S.

### Petri Dish Handling Operation Relating to the Measuring Process

The automatic measuring apparatus 1 is configured as above. The measuring process in the automatic measuring apparatus 1 and a series of Petri dish handling operations relating thereto will be explained next.

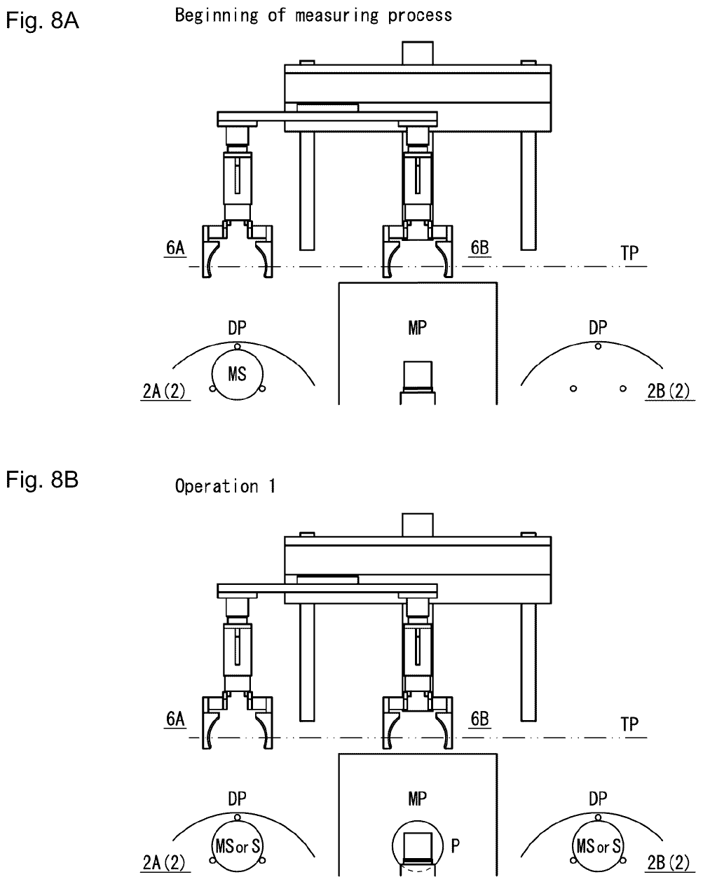

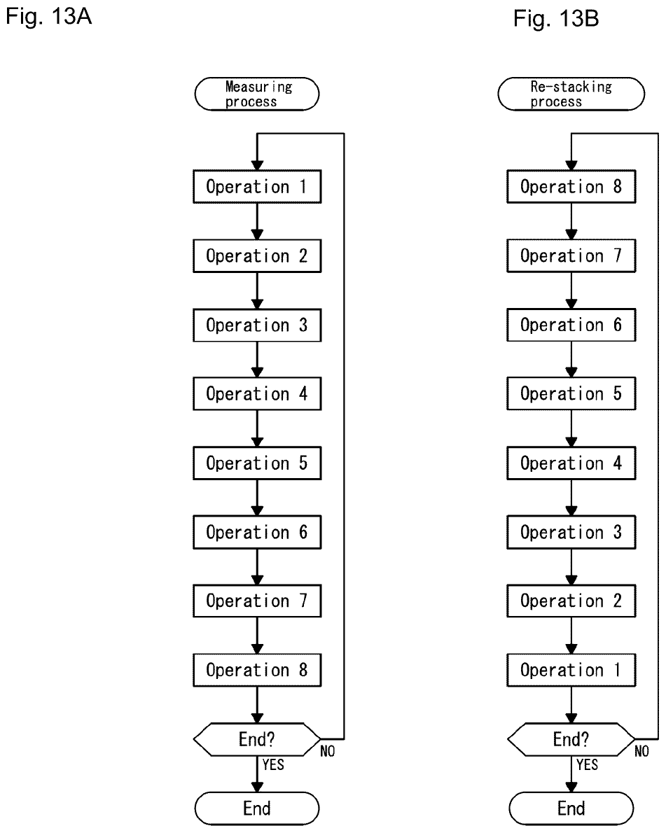

As illustrated in FIG. 13A, in the series of Petri dish handling operations, Operations 1 to 8 are performed repeatedly as shown in FIG. 8B to FIG. 12A, beginning with the state shown in FIG. 8A. Repetition is performed until all of the Petri dishes S, . . . of a multi-stack of Petri dishes MS of one magazine 22 at the first Petri dish stacking apparatus 2A are finished with the measuring process, and are accommodated in the magazine 22 of the corresponding magazine number at the second Petri dish stacking apparatus 2B. When the measuring process of one multi-stack of Petri dishes MS ends, rotary tables 20 in the respective first Petri dish stacking apparatus 2A and second Petri dish stacking apparatus 2B are angularly displaced by one pitch, and indexing of the next magazine number is performed. Subsequently, repetition is performed until all of the object multi-stack of Petri dishes MS, . . . at the first Petri dish stacking apparatus 2A are finished with the measuring process, and are accommodated in the magazine 22 of the corresponding magazine number at the second Petri dish stacking apparatus 2B. There may also be one object multi-stack of Petri dishes MS. In that case, Operations 1 to 8 shown in FIG. 8B to FIG. 12A are performed once, and then the operation shifts to a re-stacking process mentioned below. In the present embodiment, since the entire Petri dish stacking apparatus 2 can accommodate 105 Petri dishes S, a continuous automatic measuring process can be performed on a maximum of 105 Petri dishes S.

As illustrated in FIG. 8B, in Operation 1, the first gripping apparatus 6A and the second gripping apparatus 6B move so that the first gripping apparatus 6A is located on a line extending in the Y-direction from the center point of the delivery position DP of the first Petri dish stacking apparatus 2A and at the traversing position TP, and so that the second gripping apparatus 6B is located on a line extending in the Y-direction from the center point of the measuring position MP and at the traversing position TP.

In FIG. 8B, the drawings of the Petri dishes S, the multi-stack of Petri dishes MS or the plate P of the Petri dish S at the measuring position MP and the delivery position DP of the second Petri dish stacking apparatus 2B are described. Meanwhile, this expresses a state after which the repetition was progressed and the Petri dishes S were transferred to the delivery position DP of the second Petri dish stacking apparatus 2B. At the beginning of the measuring process, the Petri dishes S, the multi-stack of Petri dishes MS and the plate P of the Petri dish S are not present at these locations.

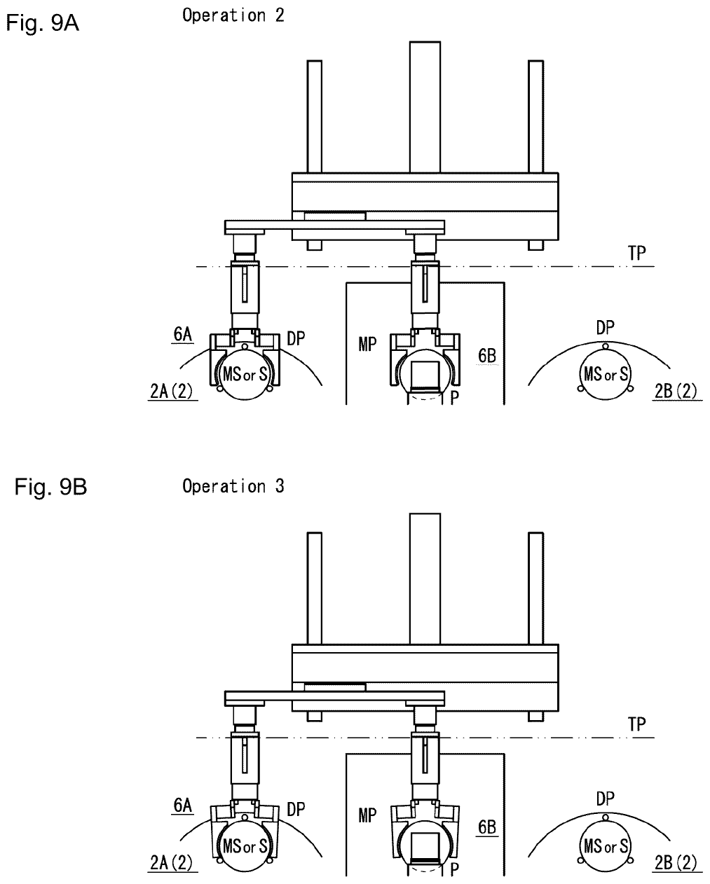

As illustrated in FIG. 9A, in Operation 2, the first gripping apparatus 6A and the second gripping apparatus 6B move so as to advance forward, and move so that the first gripping apparatus 6A is located at the delivery position DP of the first Petri dish stacking apparatus 2A, and so that the second gripping apparatus 6B is located at the measuring position MP.

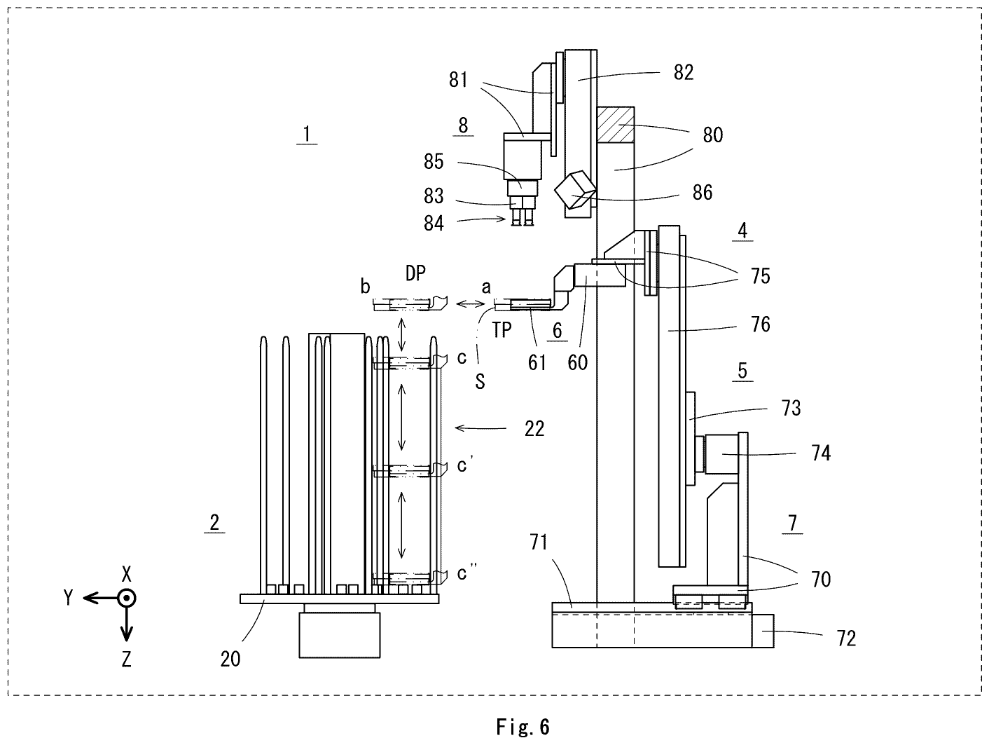

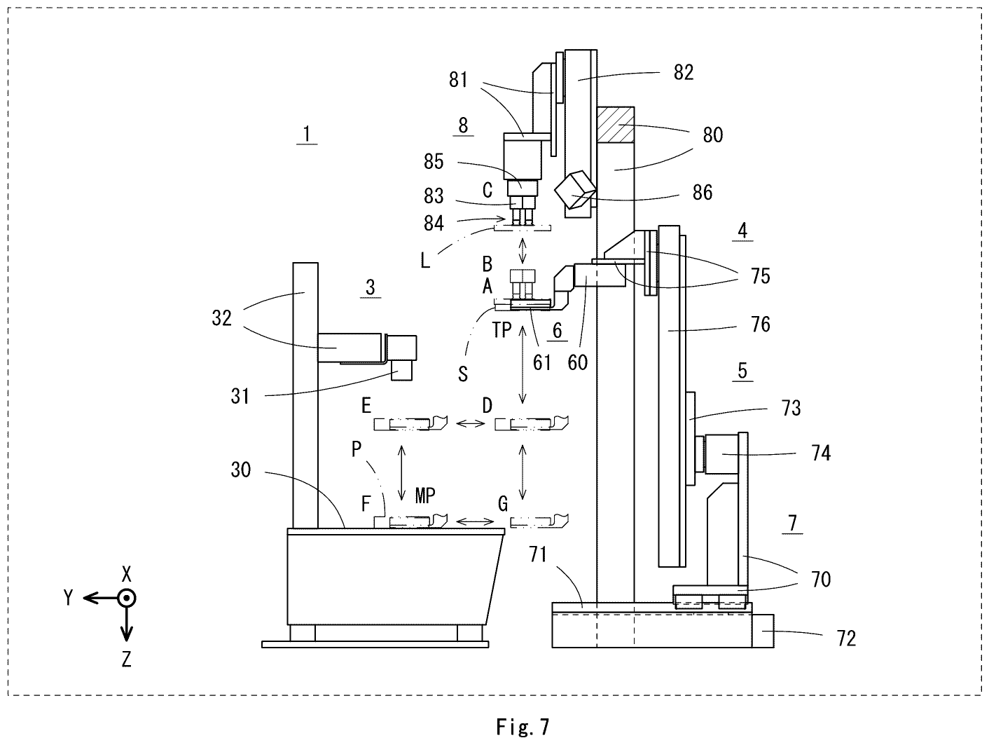

Here, upon shifting from Operation 1 to Operation 2, as illustrated in FIG. 6, the first gripping apparatus 6A moves from Position a so as to arrive at Position c to c″ (position at the height of the uppermost Petri dish S of the multi-stack of Petri dishes MS) via Position b. Moreover, as illustrated in FIG. 7, the second gripping apparatus 6B moves from Position A so as to arrive at Position F via Position D and Position E.

As illustrated in FIG. 9B, in Operation 3, the first gripping apparatus 6A holds the uppermost Petri dish S of the multi-stack of Petri dishes MS. Moreover, if a plate P of the Petri dish S is present at the measuring position MP, the second gripping apparatus 6B holds this plate.

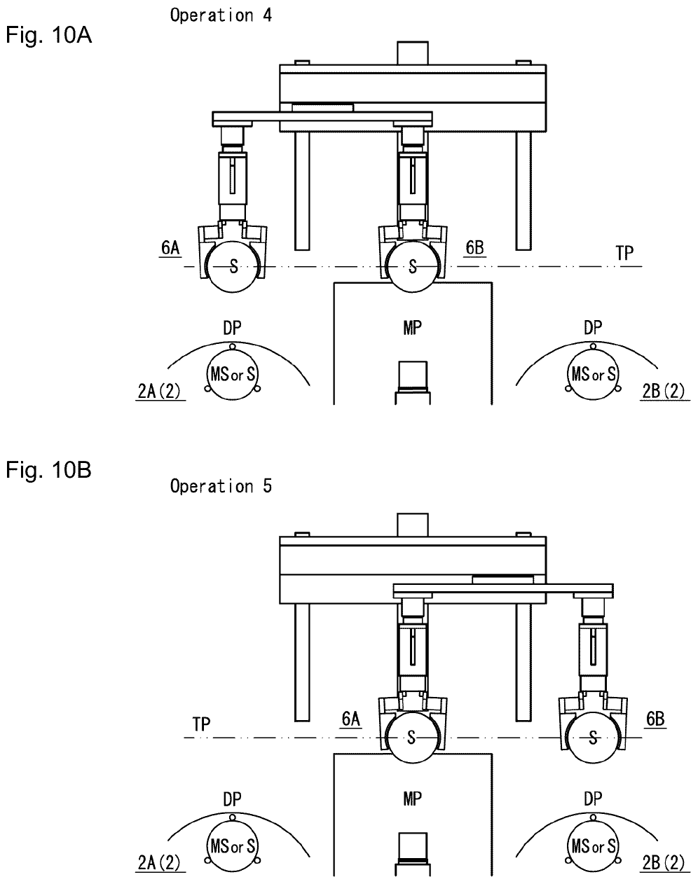

As illustrated in FIG. 10A, in Operation 4, the first gripping apparatus 6A and the second gripping apparatus 6B move so as to retract, and move so that the first gripping apparatus 6A is located on a line extending in the Y-direction from the center point of the delivery position DP of the first Petri dish stacking apparatus 2A and at the traversing position TP, and so that the second gripping apparatus 6B is located on a line extending in the Y-direction from the center point of the measuring position MP and at the traversing position TP.

Here, upon shifting from Operation 3 to Operation 4, as illustrated in FIG. 6, the first gripping apparatus 6A moves from Position c to c″ so as to return to Position a via Position b. Moreover, as illustrated in FIG. 7, the second gripping apparatus 6B moves from Position F so as to return to Position A via Position G. If a Petri dish S is present at the second gripping apparatus 6B, the head 83 of the Petri dish lid removing/attaching apparatus 8 descends from Position C to Position B and releases the lid L of the Petri dish S at Position A, after which this head 83 then ascends from Position B to Position C. The lid L is thereby put onto the Petri dish S.

As illustrated in FIG. 10B, in Operation 5, the first gripping apparatus 6A and the second gripping apparatus 6B move so as to traverse, and move so that the first gripping apparatus 6A is located on a line extending in the Y-direction from the center point of the measuring position MP and at the traversing position TP, and so that the second gripping apparatus 6B is located on a line extending in the Y-direction from the center point of the delivery position DP of the second Petri dish stacking apparatus 2B and at the traversing position TP.

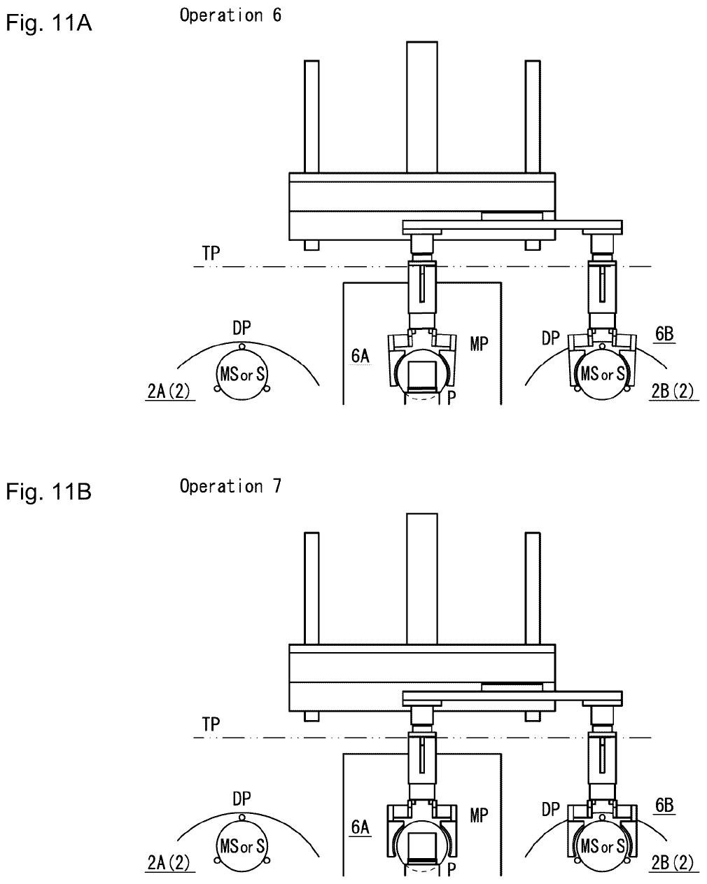

As illustrated in FIG. 11A, in Operation 6, the first gripping apparatus 6A and the second gripping apparatus 6B move so as to advance forward, and move so that the first gripping apparatus 6A is located at the measuring position MP, and so that the second gripping apparatus 6B is located at the delivery position DP of the second Petri dish stacking apparatus 2B.

Here, upon shifting from Operation 5 to Operation 6, as illustrated in FIG. 7, the first gripping apparatus 6A moves from Position A so as to arrive at Position F via Position D and Position E. Since a Petri dish S is present at the first gripping apparatus 6A, the head 83 of the Petri dish lid removing/attaching apparatus 8 descends from Position C to Position B and performs suction on the lid L of the Petri dish S at Position A, after which this head 83 then ascends from Position B to Position C. This is thereby in a state of which the lid L is removed from the Petri dish S, and only the plate P of the Petri dish S is present at the first gripping apparatus 6A. Moreover, as illustrated in FIG. 6, the second gripping apparatus 6B moves from Position a so as to arrive at Position c to c″(position at a height of one Petri dish higher than the uppermost Petri dish S) via Position b.

As illustrated in FIG. 11B, in Operation 7, the first gripping apparatus 6A releases the plate P of the Petri dish S at the measuring position MP. Thereby, from that moment until the next Operation 2, an imaging process can be performed by means of the imaging apparatus 31 of the colony measuring apparatus 3. Moreover, the second gripping apparatus 6B releases a Petri dish S upon another Petri dish S which was already transferred. A multi-stack of Petri dishes MS is thereby configured in the magazine 22 located at the delivery position DP of the second Petri dish stacking apparatus 2B.

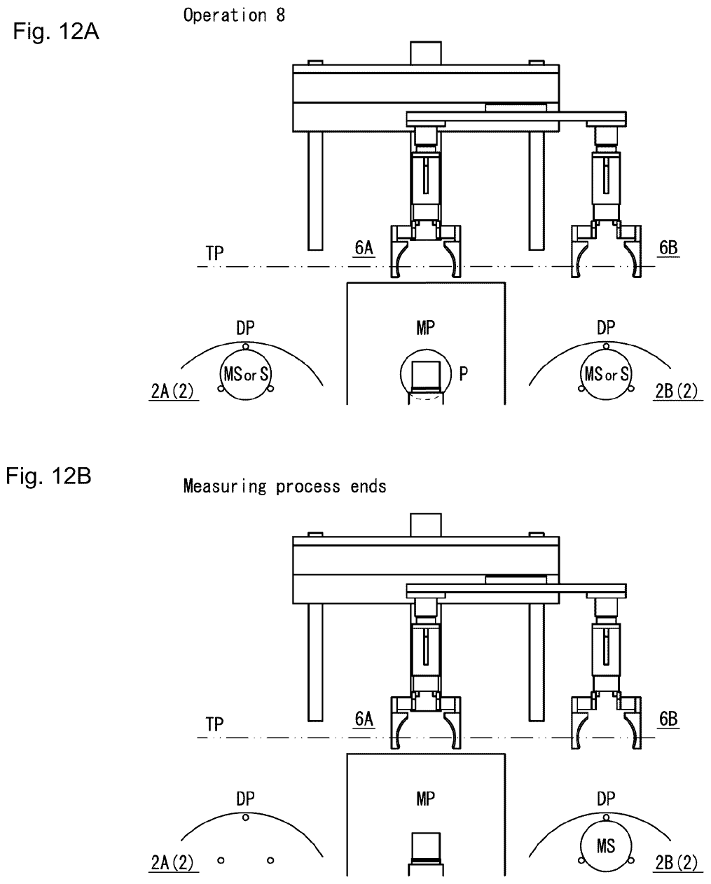

As illustrated in FIG. 12A, in Operation 8, the first gripping apparatus 6A and the second gripping apparatus 6B move so as to retract, and move so that the first gripping apparatus 6A is located on a line extending in the Y-direction from the center point of the measuring position MP and at the traversing position TP, and so that the second gripping apparatus 6B is located on a line extending in the Y-direction from the center point of the delivery position DP of the second Petri dish stacking apparatus 2B and at the traversing position TP.

In FIG. 12A, the drawings of the Petri dishes S, the multi-stack of Petri dishes MS or the plate P of the Petri dish S at the delivery position DP and the measuring position MP of the first Petri dish stacking apparatus 2A are described. Meanwhile, this expresses a state in which the Petri dishes S in the midst of repetition still remain at the first Petri dish stacking apparatus 2A. When the measuring process ends, the Petri dishes S, the multi-stack of Petri dishes MS and the plate P of the Petri dish S are not present at these positions.

Here, upon shifting from Operation 7 to Operation 8, as illustrated in FIG. 7, the first gripping apparatus 6A moves from Position F so as to return to Position A via Position G. Moreover, as illustrated in FIG. 6, the second gripping apparatus 6B moves from Position c to c″ so as to return to Position a via Position b.

As illustrated in FIG. 12B, with the repetition of the above Operation 1 to Operation 8 having ended, the multi-stack of Petri dishes MS is no longer accommodated in the magazine 22 located at the delivery position DP of the first Petri dish stacking apparatus 2A, all the Petri dishes S, . . . are finished with the measuring process, and a multi-stack of Petri dishes MS whose measuring process has completed is configured in the magazine 22 located at the delivery position DP of the second Petri dish stacking apparatus 2B. Alternatively, after the measuring process of the last Petri dish S of the multi-stack of Petri dishes MS has ended, the last Petri dish S need not be transferred to the magazine 22 located at the delivery position DP of the second Petri dish stacking apparatus 2B, but may be directly returned to the magazine 22 located at the delivery position DP of the first Petri dish stacking apparatus 2A. In this case, following the last Petri dish S, the below-mentioned re-stacking process is performed on the multi-stack of Petri dishes MS which does not include the last Petri dish S and which is accumulated in the magazine 22 located at the delivery position DP of the second Petri dish stacking 2B.

### Re-Stacking Process

Incidentally, the measuring process is continuously performed with a pre-determined time pitch (equal pitch and index change pitch), in order to see the extent of colony growth. Therefore, a multi-stack of Petri dishes MS configured at the second Petri dish stacking apparatus 2B must be returned to the original magazine 22 of the first Petri dish stacking apparatus 2A. However, as illustrated in FIG. 14A and FIG. 14B, the vertical order of the Petri dishes S of the multi-stack of Petri dishes MS is reversed with that of the original multi-stack of Petri dishes MS, and hence if (a person) were to return this as it is, the order of the measuring process would end up being reversed in the subsequent measuring process. Therefore, the re-stacking process shown in FIG. 13B is automatically executed at a predetermined timing after the measuring process.

In the re-stacking process, the aforementioned Operations 1 to 8 are repeatedly performed in reverse order, as a return operation. Even by repeating Operations 1 to 8 in reverse order, a relay operation is performed on the Petri dishes S from the second gripping apparatus 6B to the first gripping apparatus 6A at the measuring position MP. However, of course, the removing/attaching process of the lid L of the Petri dish S and the imaging process of the plate P of the Petri dish S is not performed. Therefore, the total operating time is shorter than the total operating time during the measuring process.

In the timing of the re-stacking process, one example includes the point in time when, based on the multi-stack of Petri dishes MS of one magazine 22 at the first Petri dish stacking apparatus 2A, the multi-stack of Petri dishes MS is configured in the magazine 22 of the corresponding magazine number at the second Petri dish stacking apparatus 2B; namely, the point in time before the multi-stack of Petri dishes MS of the next magazine 22 undergo the measuring process. Another example includes the point in time when, based on all the object multi-stack of Petri dishes MS, . . . at the first Petri dish stacking apparatus 2A, all the multi-stack Petri dishes MS, . . . are configured at the second Petri dish stacking apparatus 2B.

By executing the re-stacking process in this way, the multi-stack of Petri dishes MS configured at the second Petri dish stacking apparatus 2B is automatically returned to the first Petri dish stacking apparatus 2A, where the vertical order of the Petri dishes S is made to be the same as the original multi-stack of Petri dishes MS. Therefore, the reversing of the order of the measuring process in a multi-stack of Petri dishes MS can be prevented, and the order of the measuring process from the next time onwards can also be made the same. Moreover, in a measuring process continuously performed with a pre-determined time pitch, the order of the measuring process each time is always the same. Therefore, the takt time can be made to become fixed.

### Scope of the Invention

The present invention is not limited to the aforementioned embodiment, and various changes can be made within a scope which does not deviate from the gist of the present invention.

In the aforementioned embodiment, one Petri dish at a time is taken out from the top of the multi-stack of Petri dishes MS at the first Petri dish stacking apparatus 2A and the Petri dish S is disposed sequentially on the top at the second Petri dish stacking apparatus 2B to form a multi-stack of Petri dishes MS. However, the present invention is not limited to this embodiment. For example, the present invention may be configured so that one Petri dish S at a time is taken out from the bottom of the multi-stack of Petri dishes MS at the first Petri dish stacking apparatus 2A and the Petri dish S is disposed sequentially on the bottom at the second Petri dish stacking apparatus 2B to form a multi-stack of Petri dishes MS.

Moreover, in the aforementioned embodiment, by synchronizing the first Petri dish stacking apparatus 2A and second Petri dish stacking apparatus 2B, the Petri dishes S accommodated in the magazine 22 at the first Petri dish stacking apparatus 2A are accommodated in the magazine 22 of the corresponding magazine number in the second Petri dish stacking apparatus 2B. However, the present invention is not limited to this embodiment. The first Petri dish stacking apparatus 2A and the second Petri dish stacking apparatus 2B need not be synchronized, but any magazine 22 may also be selected from amongst the plurality of the magazines 22, . . . of the second Petri dish stacking apparatus 2B.

Moreover, in the aforementioned embodiment, the second Petri dish stacking apparatus 2B provided with a plurality of the magazines 22, . . . is used as a temporary placement section for temporarily placing the Petri dishes S transferred from the first Petri dish stacking apparatus 2A. However, the present invention is not limited to this embodiment. In the aforementioned example of the re-stacking process (the example in which the re-stacking process is performed at a magazine 22 unit), the number of magazines 22 of the second Petri dish stacking apparatus 2B may also be 1.

Moreover, the temporary placement section need not be at the second Petri dish stacking apparatus 2B, but may also be set in an empty magazine 22 of the first Petri dish stacking apparatus 2A. In this case, the measuring process and the re-stacking process are combined, and a series of operations are repeatedly performed until the measuring process of all the Petri dishes S, . . . ends, where the series of operations includes: Operation 1→Operation 2→Operation 3→Operation 4→Operation 5→Operation 6→Operation 7→Operation 8→Operation 7→Operation 6→Operation 5→Operation 4 (indexing of a magazine number which was set as a temporary placement section in between Operation 4 until the end of Operation 4)→Operation 3 →Operation 2→Operation 1→indexing of the original magazine number. In this case, the Petri dish S merely goes back and forth between the delivery position DP of the first Petri dish stacking apparatus 2A and the measuring position MP. Therefore, the second Petri dish stacking apparatus 2B is not indispensable.

Moreover, in the aforementioned embodiment, the predetermined process is the measuring process. However, the present invention is not limited to this embodiment. A predetermined process may also be another process.

Moreover, in the aforementioned embodiment, the gripping apparatus 6 is provided with two gripping apparatuses: the first gripping apparatus 6A and the second gripping apparatus 6B. However, the present invention is not limited to this embodiment. There may be only one gripping apparatus where the Petri dishes S may also be transferred by means of one gripping apparatus between the first Petri dish stacking apparatus 2A, the colony measuring apparatus 3 and the second Petri dish stacking apparatus 2B.

Moreover, in the aforementioned embodiment, a three-axis XYZ-orthogonal drive apparatus 7 is used as the drive apparatus of the Petri dish transferring apparatus 5. However, the present invention is not limited to this embodiment. All kinds of known apparatuses may be adopted as a drive apparatus. For example, if utilizing a robot arm as the drive apparatus, in the re-stacking process, access to the measuring position MP, and the relay operation of a Petri dish S from the second gripping apparatus 6B to the first gripping apparatus 6A at the measuring position MP can be eliminated.

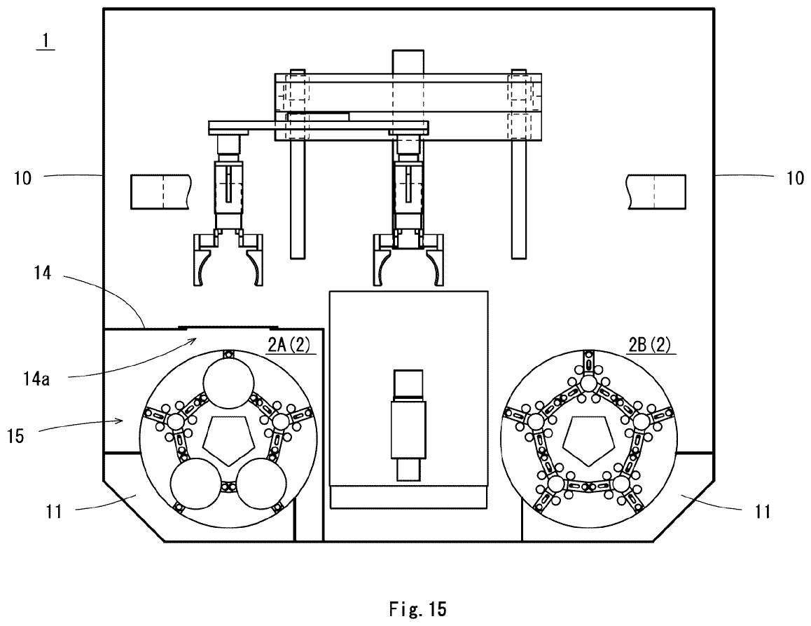

Moreover, as illustrated in FIG. 15, an incubator 15 may also be installed. The incubator 15 is a structure which encloses the first Petri dish stacking apparatus 2A by a cover of the enclosure 10 (exterior plate) and a partition wall 14 provided with an openable-and-closable gate 14a. Alternatively, the incubator 15 may also be a structure which encloses the first Petri dish stacking apparatus 2A by an air curtain. Alternatively, the entire interior of enclosure 10 may also be an incubator.

### Other Inventions

According to the apparatus configuration according to the above invention, the following ways of usage are possible.

### Rotation Correction Process in the Measuring Process

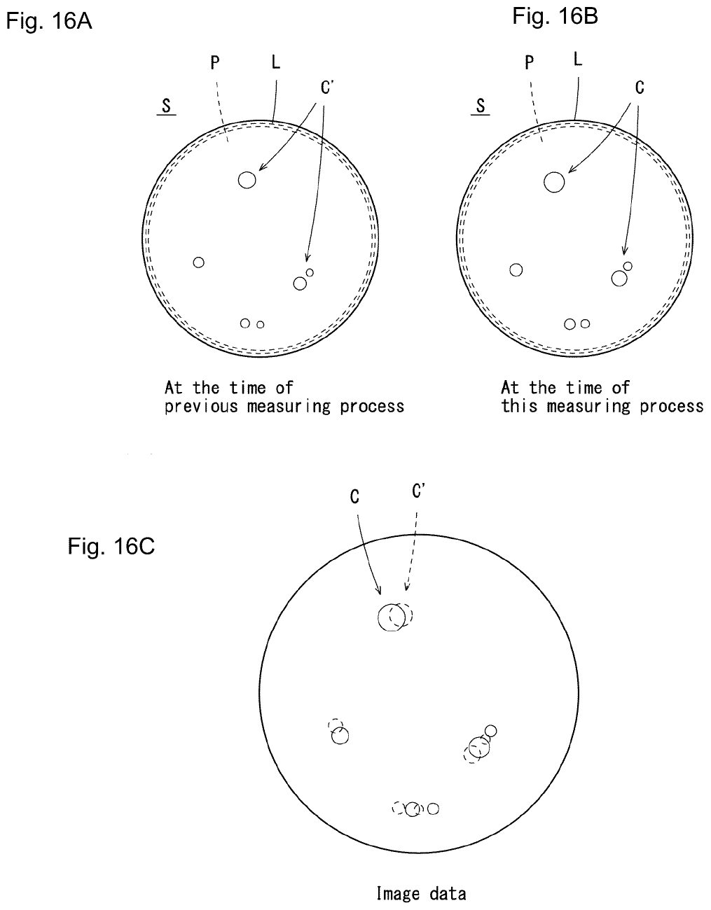

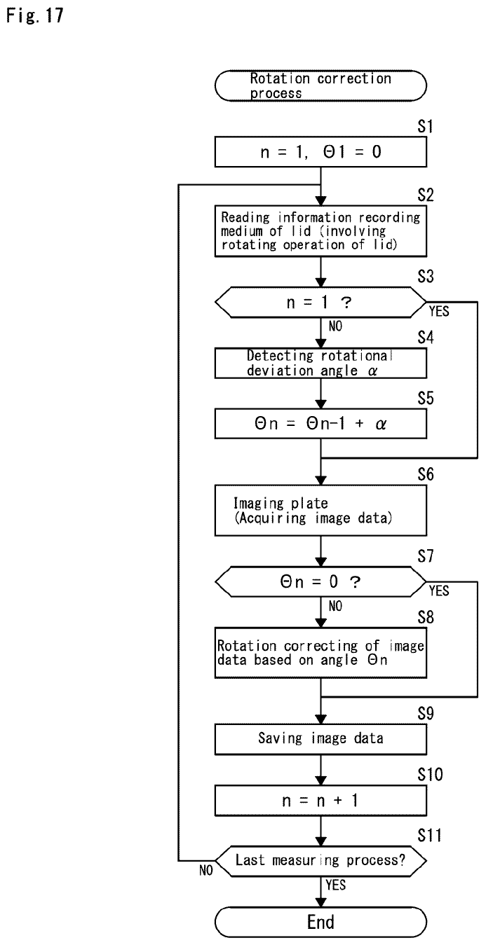

During the handling of a Petri dish S, it cannot be denied that a rotational deviation about the vertical axis may occur at the Petri dish S, for example, at the timing of holding and releasing the Petri dish S by means of the gripping apparatus 6. As illustrated in FIG. 16A and 16B, the rotational deviation is revealed in the measuring process from the second time onwards after the first-time measuring process. Then, as illustrated in FIG. 16C, when a rotational deviation occurs, it also occurs in the image of the plate P which was captured at the measuring position MP, and by extension in an image for the measuring process. Even if a rotational deviation occurs in the image for the measuring process, there is no mis-counting of the number of colonies C, C′. However, a high level of measurement results cannot be obtained due to the loss of correlation of the same colonies C, C′. Thus, the rotation correction process shown in FIG. 17 may be utilized.

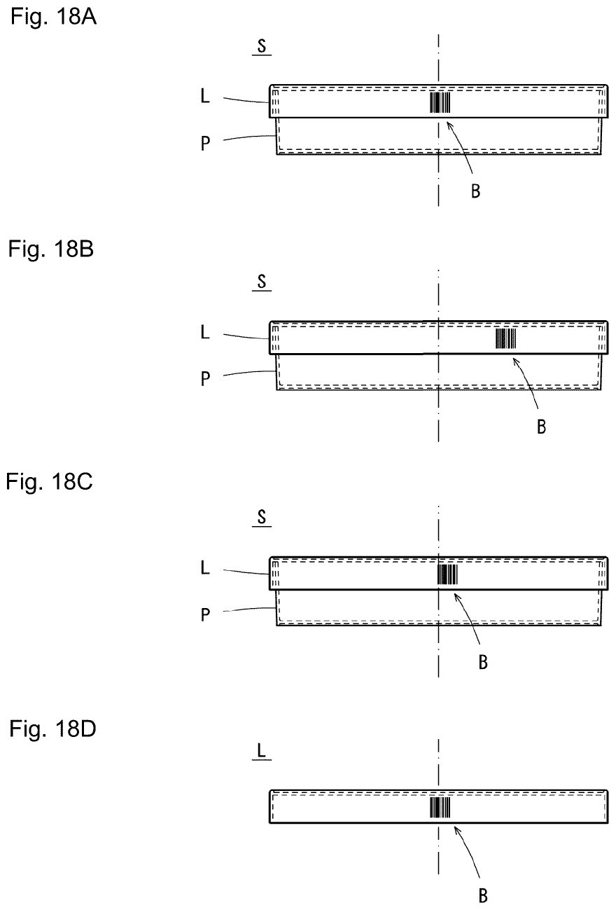

First, in Step 1, an n-value of a counter is set to 1, and the value of n=1 of an angular amount On is set to 0 (zero). Next, in Step 2, an information recording medium such as a bar code, a QR code (TM) and an RF-ID tag assigned to the upper surface or circumferential side surface of the lid L is read by means of the reading device 86, in a state of which the lid L was taken off from a Petri dish S by means of the Petri dish lid removing/attaching apparatus 8. In the first-time measuring process, as illustrated in FIG. 18B, if an information recording medium B is not located at the center for reading (when a person stacks Petri dishes S to form a multi-stack of Petri dishes MS, and then places the multi-stack of Petri dishes MS at the first Petri dish stacking apparatus 2A, he/she is not concerned with the location of the information recording medium B, and thus the information recording medium B is basically not located at the center for reading), by means of the rotation of the head 83 of the Petri dish lid removing/attaching apparatus 8 as illustrated in FIG. 18D, the lid L is rotated so that the information recording medium B is located at the center for reading. After the measuring process, the lid L is put onto the plate P, remaining in the state of having been rotated.

Next, in Step 3, when n=1 (when Step 3 is YES); namely, for the first-time measuring process, Step 6 is executed. In Step 6, the plate P is photographed at the measuring position MP, and an image data of the plate P is acquired. Next, in Step 7, if the angular amount On is 0 (when Step 7 is YES), Step 9 is executed. In Step 9, the image data and an image for the measuring process generated based on this image data are saved to the memory unit of the control circuit 13.

Next, in Step 10, a counter is counted up, and the n-value becomes 2 or more. If this is not the final measuring process (when Step 11 is NO), the rotation correction process shifts to Step 2, and the measuring process from the second time onwards is begun.

In Step 2, the information recording medium B is read by means of the reading device 86. However, even for the measuring process from the second time onwards, if a rotational deviation has occurred at a Petri dish S due to the handling operation until then, as illustrated in FIG. 18C, the information recording medium B is once again deviated from the center for reading due to rotational deviation having occurred at the lid L. In this case, similarly, by means of the rotation of the head 83 of the Petri dish lid removing/attaching apparatus 8, as illustrated in FIG. 18D, the lid L is rotated so that the information recording medium B is located at the center for reading. After the measuring process, the lid L is put onto the plate P, remaining in the state of having been rotated. Namely, relative angular displacement occurs between the plate P and the lid L.

In Step 3, when the n-value is 2 or more (when Step 3 is NO); namely, for the measuring process from the second time onwards, the rotational deviation angle α of the lid L is detected in Step 4. This is made possible by acquiring information on the rotated angular amount of the head 83, from an encoder with which the actuator 85 is provided. In Step 2, the rotational deviation of the lid L was corrected by the lid L having been rotated by angle a in the opposite direction.

Next, in Step 5, an angular amount On is calculated. In the angular amount θn, the rotational deviation angle a of the lid L is added to the previous angular amount θn−1. Namely, the angular amount On is expressed by a relative value of the rotational deviation angle based on the previous angular amount θn−1.

Next, in Step 6, the image data of the plate P is acquired. Meanwhile, if a rotational deviation has occurred at a Petri dish S due to the handling operation until then (if Step 7 is NO), as mentioned above, a rotational deviation also occurs in the image of the plate P, and by extension in the image for the measuring process. Thus, in Step 8, the control circuit 13 subjects the rotation correction process to the image of the plate P, the image for the measuring process, or any image data of intermediate images between those images. The rotation correction process rotates the image data by an angular amount On in the opposite direction.

Then, in Step 9, the image data having undergone the rotation correction process is saved to the memory unit of the control circuit 13. These series of processes are repeated until all the Petri dishes S, . . . of a multi-stack of Petri dishes MS are finished with the measuring process (when Step 11 is YES).

As described above, the rotation correction process of the image data is executed, using the information recording medium B assigned to the lid L as an alignment marking. Therefore, rotational deviation can be eliminated in all images for the measuring process, and the images can be configured in a state where they are easily compared with each other. Moreover, correlation of the same colonies are therefore maintained, and a higher level of measuring and analysis can be performed.

Moreover, regardless of whether or not the rotation correction process of Step 8 was performed, the rotating operation of Step 2 is performed on all the lids L, . . . of the Petri dishes S, . . . of a multi-stack of Petri dishes MS. Therefore, the information recording medium B, . . . of all the lids L, . . . in the multi-stack of Petri dishes MS can be arranged in one vertical row.

The rotating operation of Step 2 of the lid L may also be such that the lid L is rotated in one direction, and the rotation of the lid L is stopped at the point in time when the reading device 86 has read the information recording medium B. Thereby, by acquiring the stop location information of the lid L from last time and the stop location information of the lid L from this time from the encoder of the actuator 85, the rotational deviation angle a of the lid L can also be detected.

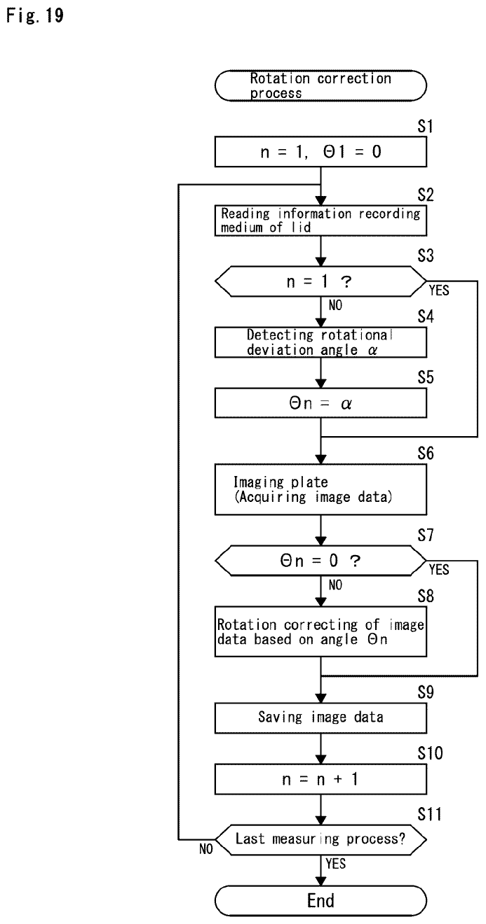

Moreover, if the reading device 86 (and the control circuit 13) can detect the rotational deviation angle a of the lid L by utilizing image processing such as image matching etc. on the information recording medium B, then a rotation correction process as shown in FIG. 19 where the rotating operation of the lid L is not performed, may also be utilized. In this case, the angular amount On is expressed by an absolute value.

Moreover, the detection criterion of the rotational deviation angle a of the lid L need not be the information recording medium B, but may also be any display medium such as a number, lettering, marking etc. assigned to the lid L.

Moreover, the information recording medium or display medium may also be assigned to the circumferential side surface of the plate P of the Petri dish S. In this case, a rotating robot for rotating the plate P is installed. In each measuring process, the rotating robot suitably rotates the Petri dishes S to make the angles of the information recording medium and display medium identical, and thereby Steps 4, 5, 7 and 8 of FIG. 17 become unnecessary.

One example of an automatic measuring apparatus is provided with:
a placement section in which containers each comprising a lid and a plate can be placed, where an information recording medium or display medium has been assigned to the lid, and where a sample has been put into the plate,
a container transferring apparatus which can transfer the container,
an imager for capturing an image of the sample put into the plate of the container,
a rotating apparatus for rotating the lid of the container,
a reader for reading the information recording medium or display medium assigned to the lid of the container, and
a control circuit for controlling the container transferring apparatus, the imager, the rotating apparatus and the reader, where
controlled by the control circuit, the container transferring apparatus takes out the container from the containers placed on the placement section in a manner of one container at a time and transfers at least the plate of the taken out container to the imager,
controlled by the control circuit, the rotating apparatus rotates the lid so that the information recording medium or display medium assigned to the lid of the taken out container is placed at a predetermined rotation angle,
controlled by the control circuit, the reader reads the information recording medium or display medium rotated to the predetermined rotation angle,
the imager captures an image of the sample of the transferred plate, and
the control circuit, when the lid of the container is rotated in the image capturing process from the second time onwards, stores the angular information of the rotation thereof, and rotates the captured image according to the rotation angle thereof.

### Cultivation Conditions Equalization Process

In the automatic measuring apparatus 1 in which a first Petri dish stacking apparatus 2A storing a multi-stack of Petri dishes MS is put under a cultivation environment in the aforementioned incubator 15 (see FIG. 15), the incubator 15 is configured and controlled so that the entire interior becomes a uniform cultivation environment. However, it cannot be denied that non-uniformity such as a temperature gradient may occur to a certain extent in the cultivation environment. If a non-uniformity occurs in the cultivation environment, variances will occur in the cultivation conditions between the Petri dishes S, . . . of the multi-stack of Petri dishes MS. Thus, the cultivation conditions equalization process can be utilized. There are two cultivation conditions equalization processes.

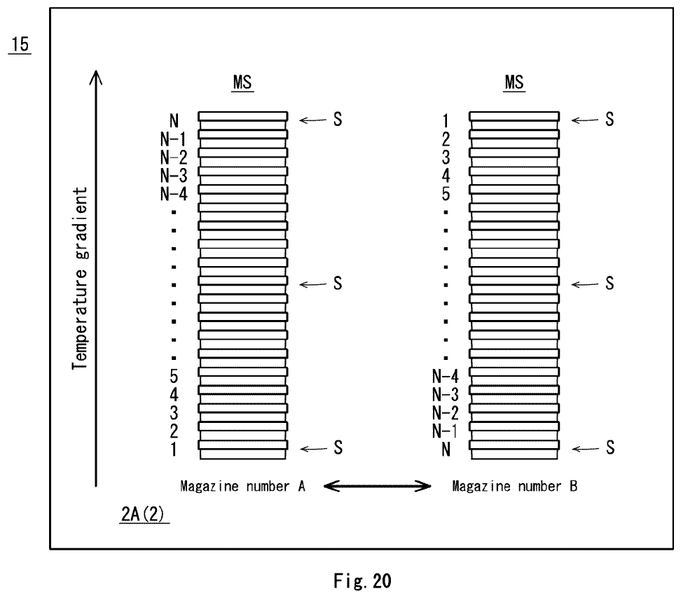

As illustrated in FIG. 20, one cultivation conditions equalization process is for the case in which a temperature gradient occurs in the vertical direction in the cultivation environment in the incubator 15. By means of the cultivation conditions equalization process, transfer of the multi-stack of Petri dishes MS is performed at appropriate time intervals. The transfer is performed, for example, between two different magazines 22, 22 of the first Petri dish stacking apparatus 2A. In this case, the transfer of the multi-stack of Petri dishes MS is repeated on the basis of a series of operations, where the series of operations includes: Operation 1 (FIG. 8B)→Operation 2 (FIG. A)→Operation 3 (FIG. 9B)→Operation 4 (FIG. 10A)→indexing of a separate magazine number→Operation 3→Operation 2→Operation 1→indexing of the original magazine number or indexing of a further separate magazine number.

Upon the transfer, the vertical order of the Petri dishes S of the multi-stack of Petri dishes MS becomes reversed with that of the original multi-stack of Petri dishes MS. Thereby, the Petri dishes S located until now at the side where the temperature is high are transferred to the side where the temperature is low; on the other hand, the Petri dishes S located until now at the side where the temperature is low are transferred to the side where the temperature is high. Therefore, by periodically repeating the transfer of the multi-stack of Petri dishes MS, the cultivation conditions of each of the Petri dishes S can be equalized.

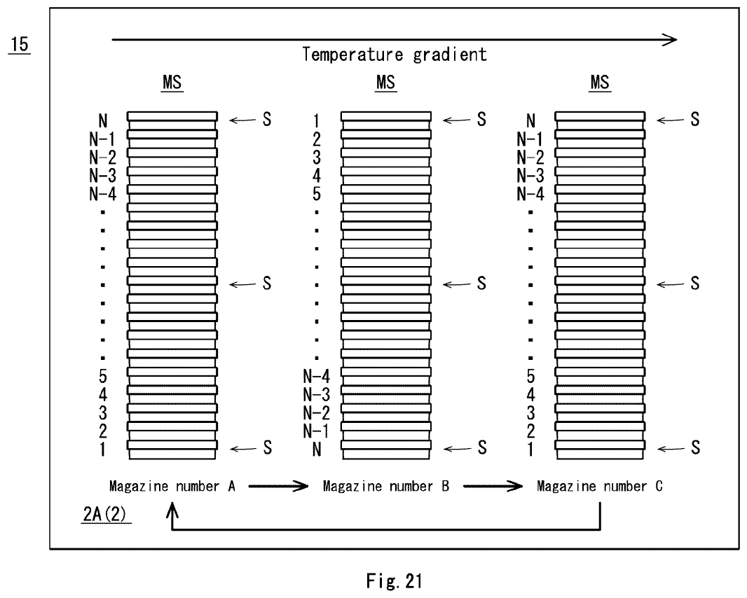

As illustrated in FIG. 21, one other cultivation conditions equalization process is for the case in which a temperature gradient occurs in the horizontal direction in the cultivation environment in the incubator 15. The content of the process is basically similar to that of the first cultivation conditions equalization process. With the second cultivation conditions equalization process, the difference is in performing a circular route (circulating) transfer of the multi-stack of Petri dishes S in multiple locations (at least two locations) using the direction of the temperature gradient as a criterion.

Upon the transfer, the multi-stack of Petri dishes MS is transferred to a location where the temperature differs. Thereby, all the Petri dishes S, . . . circulate to a plurality of locations where the temperature differs. Therefore, by periodically repeating the transfer of the multi-stack of Petri dishes MS, the cultivation conditions of each of the Petri dishes S can be equalized.

Needless to say, the cultivation conditions equalization process can also be applied to an automatic measuring apparatus 1 of which the incubator is not provided.

One example of an automatic measuring apparatus is provided with:
a placement section in which a multi-stack of containers can be placed,
an incubator for accommodating the placement section,
a container transferring apparatus which can transfer a container, and
a control circuit for controlling the container transferring apparatus, where
controlled by the control circuit, the container transferring apparatus, at predetermined time intervals, takes out the container from a top of the multi-stack of containers of the placement section in a manner of one container at a time and disposes the container sequentially on a top of another container at another location of the placement section to form a multi-stack of containers at another location, or takes out the container from a bottom of the multi-stack of containers of the placement section in a manner of one container at a time and disposes the container sequentially on a bottom at another location of the placement section to form a multi-stack of containers at another location.

### Another Usage Example 1

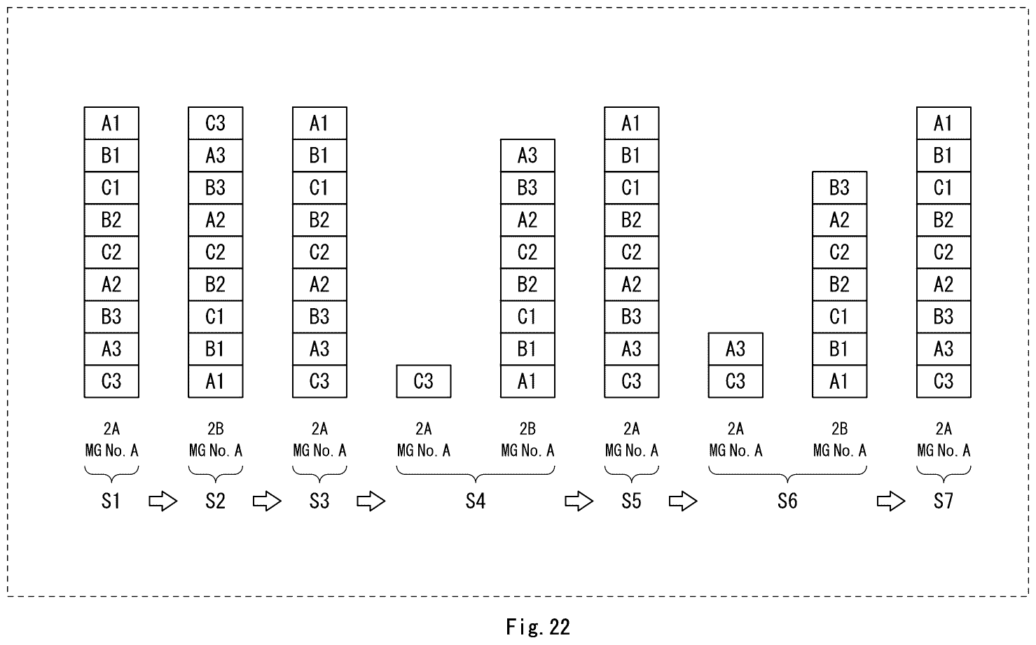

Usage Example 1 is illustrated in FIG. 22. The drawings of the Petri dishes S have been simplified in the drawings utilized with the below explanation of each usage examples. Starting from Step 1, the first-time measuring process is performed on all the Petri dishes S, . . . in Step 2. Alternatively, a colony counting process need not be performed but only an imaging process may be performed. Alternatively, the counting process and the imaging process need not be performed. Moreover, at this time, the reading process of the information recording medium assigned to the Petri dishes S is performed on all the Petri dishes S, . . . . By means of this reading process, the control circuit 13 identifies the group to which the Petri dishes S of each stack of the multi-stack of Petri dishes MS belong, and stores the identification results to a recording medium of the control circuit 13. After these processes, a multi-stack of Petri dishes MS is formed at the second Petri dish stacking apparatus 2B, in a state in which the vertical order of the Petri dishes S was reversed with that of the original multi-stack of Petri dishes MS. Therefore, the re-stacking process is performed in Step 3.

Next, in Step 4, the second-time measuring process (if the measuring process was not performed in Step 2, the first-time measuring process) is performed on the Petri dishes S (A1 to A3) of one group (Group A) of a plurality of groups (in the present example, three Groups A to C; hereinafter in the same applies to each relevant usage example). In this case, the measuring process is not performed on the Petri dishes S, . . . of the other groups (Group B and Group C). Then, when the measuring process of the last Petri dish S (A3) of one group (Group A) ends, the re-stacking process is performed in Step 5. A group is categorized by recorded information held by the information recording medium such as type of sample (microbial species etc.), time interval of the measuring process, order of the measuring process, or processing conditions of the measuring process (hereinafter in the same applies to each relevant usage example).

Next, in Step 6, the second-time measuring process (if the measuring process was not performed in Step 2, the first-time measuring process) is performed on the Petri dishes S (B1 to B3) of a separate group (Group B). In this case, the measuring process is not performed on the Petri dishes S, . . . of the other groups (Group A and Group C). Then, when the measuring process of the last Petri dish S (B3) of the separate group (Group B) ends, the re-stacking process is performed in Step 7.

Thereafter, the measuring process is performed similarly on the Petri dishes S, . . . of any one group whose predetermined measuring time has arrived. Then, when the measuring process of the last Petri dish S of the relevant group ends, the re-stacking process is performed. The time interval of the measuring process is configured to be Group A<Group B<Group C.

The Petri dishes S are not transferred to the second Petri dish stacking apparatus 2B; namely, the second Petri dish stacking apparatus 2B need not be utilized as a temporary placement section, but the Petri dishes S may also be transferred to an empty magazine 22 of the first Petri dish stacking apparatus 2A; namely, an empty magazine 22 of a separate magazine number of the first Petri dish stacking apparatus 2A may also be utilized as the temporary placement section (hereinafter similar in each relevant usage example).

### Another Usage Example 2

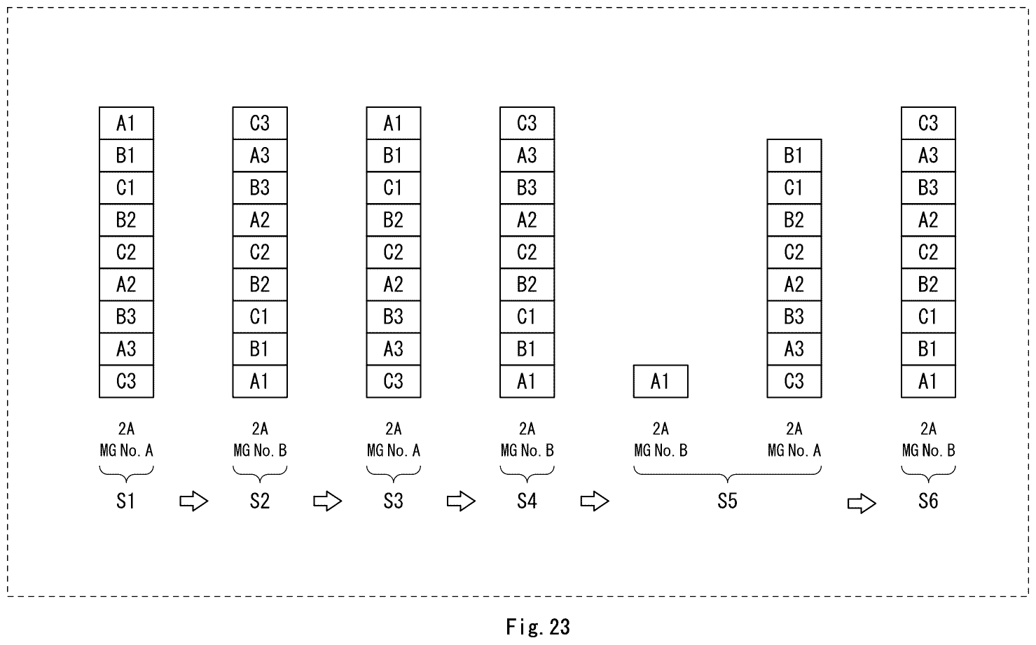

Usage Example 2 is illustrated in FIG. 23. Starting from Step 1, and the reading process of the information recording medium assigned to the Petri dishes S is performed on all the Petri dishes S, . . . in Step 2. By means of this reading process, the control circuit 13 identifies the group to which the Petri dishes S of each stack of the multi-stack of Petri dishes MS belong, and stores the identification results to a recording medium of the control circuit 13. After the reading process, a multi-stack of Petri dishes MS is formed in a state in which the vertical order of the Petri dishes S was reversed with that of the original multi-stack of Petri dishes MS, in the magazine 22 of a separate magazine number (Magazine Number B) of the first Petri dish stacking apparatus 2A. Next, in Step 3, the first-time measuring process is performed on the Petri dishes S (A1 to A3) of one group (Group A). In this case, the measuring process is not performed on the Petri dishes S, . . . of the other groups (Group B and Group C). After the measuring process, a multi-stack of Petri dishes MS is formed, in the magazine 22 of the original magazine number (Magazine Number A), in a state in which the vertical order of the Petri dishes S was reversed with that of the multi-stack of Petri dishes MS formed in the magazine 22 of a separate magazine number (Magazine Number B). Therefore, the re-stacking process is performed in Step 4.

Next, in Step 5, the first-time measuring process is performed on the Petri dishes S (B1 to B3) of a separate group (Group B). In this case, the measuring process is not performed on the Petri dishes S, . . . of the other groups (Group A and Group C). Then, when the measuring process of the last Petri dish S (B1) of the separate group (Group B) ends, the re-stacking process is performed in Step 6.

Thereafter, the measuring process is performed similarly on the Petri dishes S, . . . of any one group whose predetermined measuring time has arrived. Then, when the measuring process of the last Petri dish S of the relevant group ends, the re-stacking process is performed. The time interval of the measuring process is configured to be Group A<Group B<Group C.

### Another Usage Example 3 (Group-Based Sorting Process)

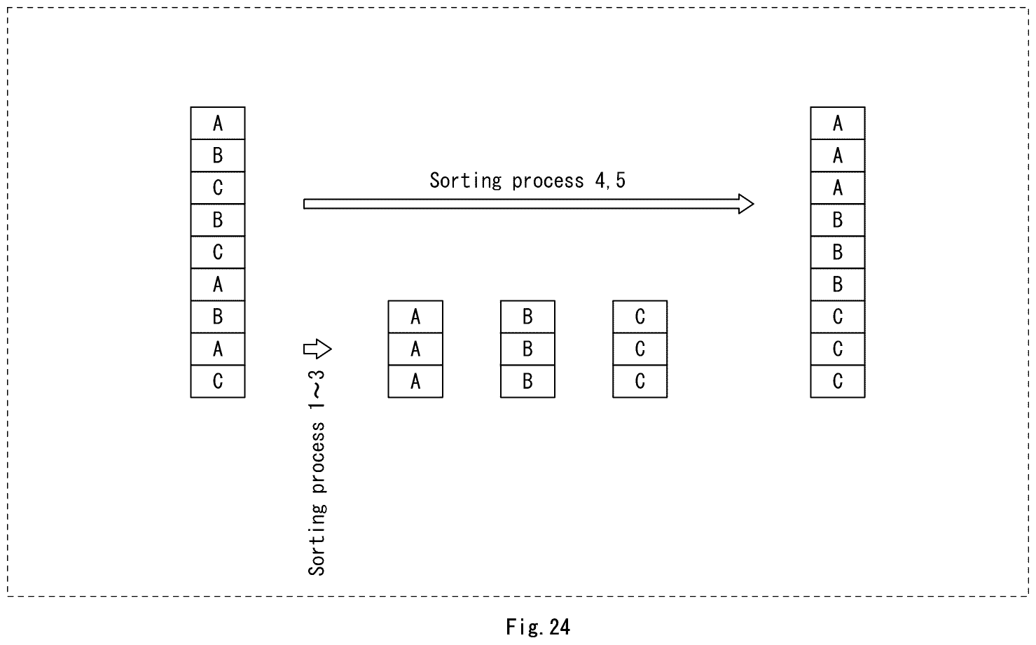

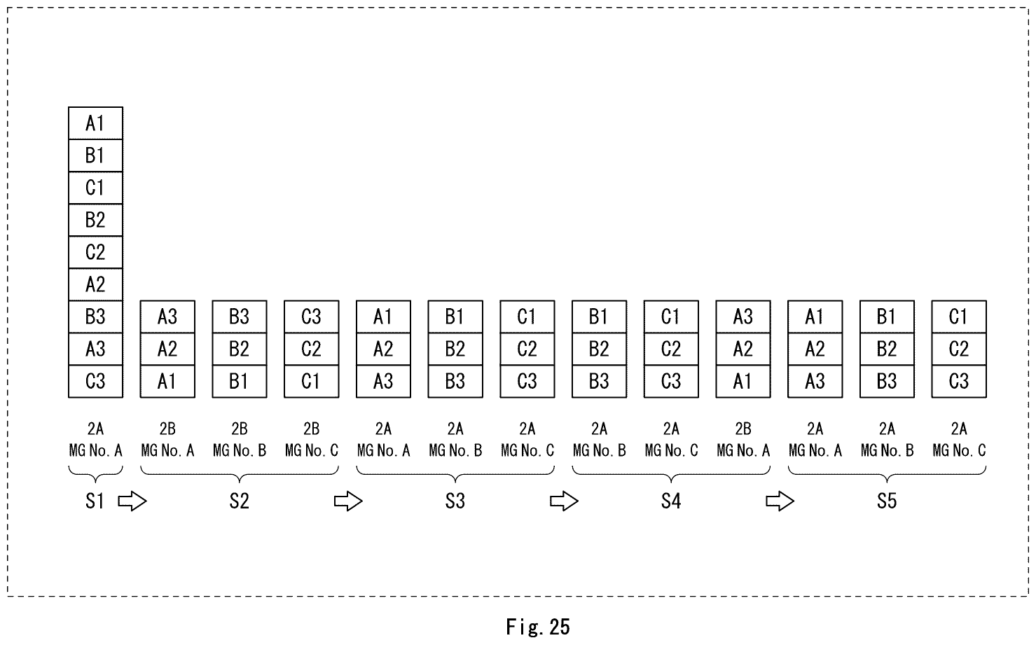

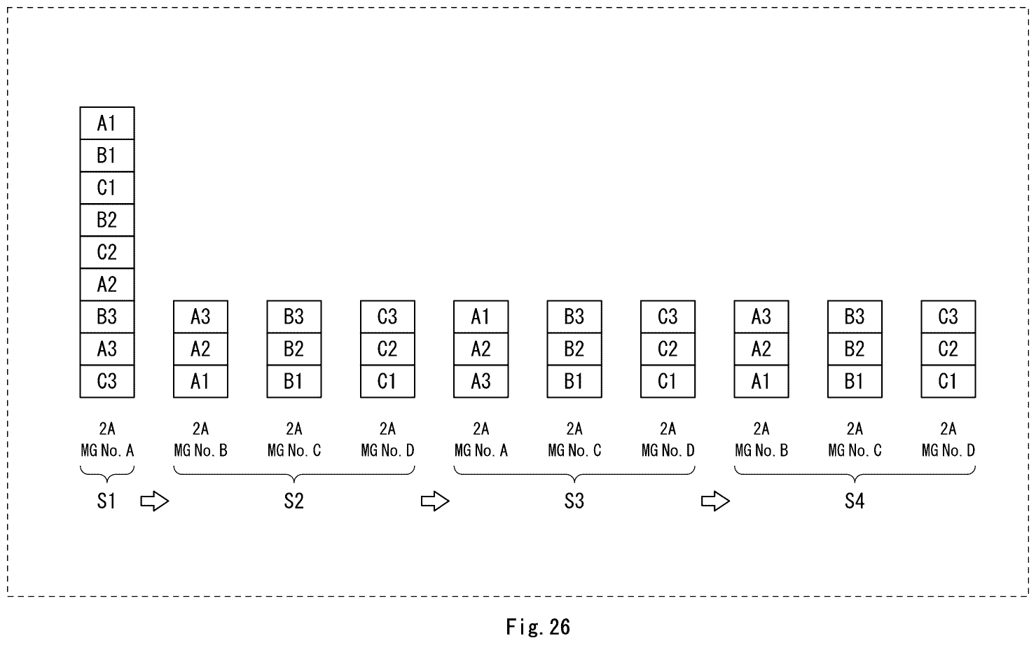

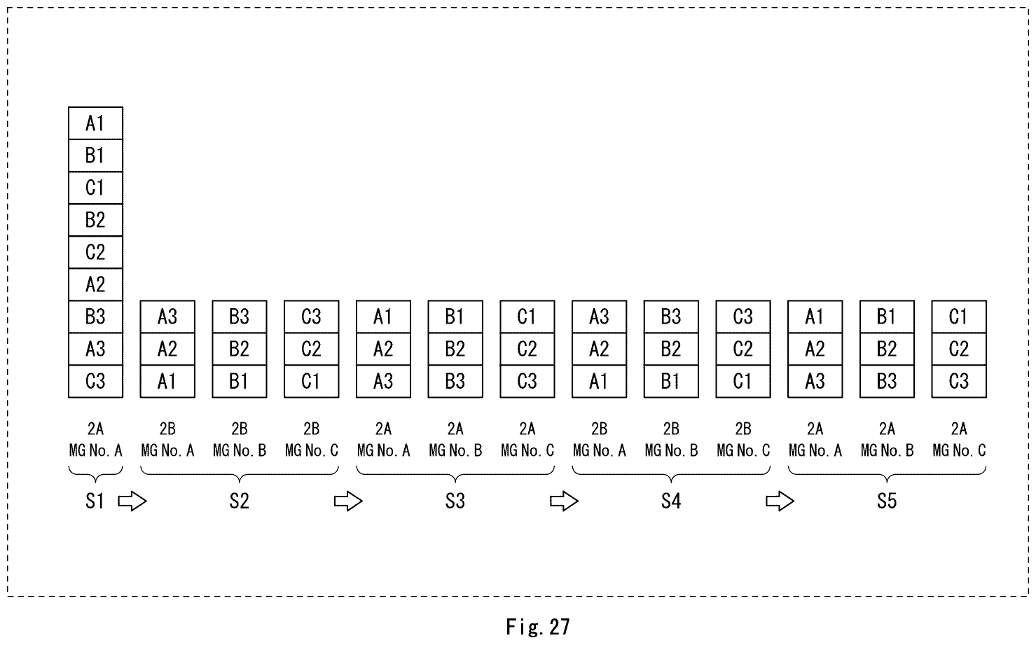

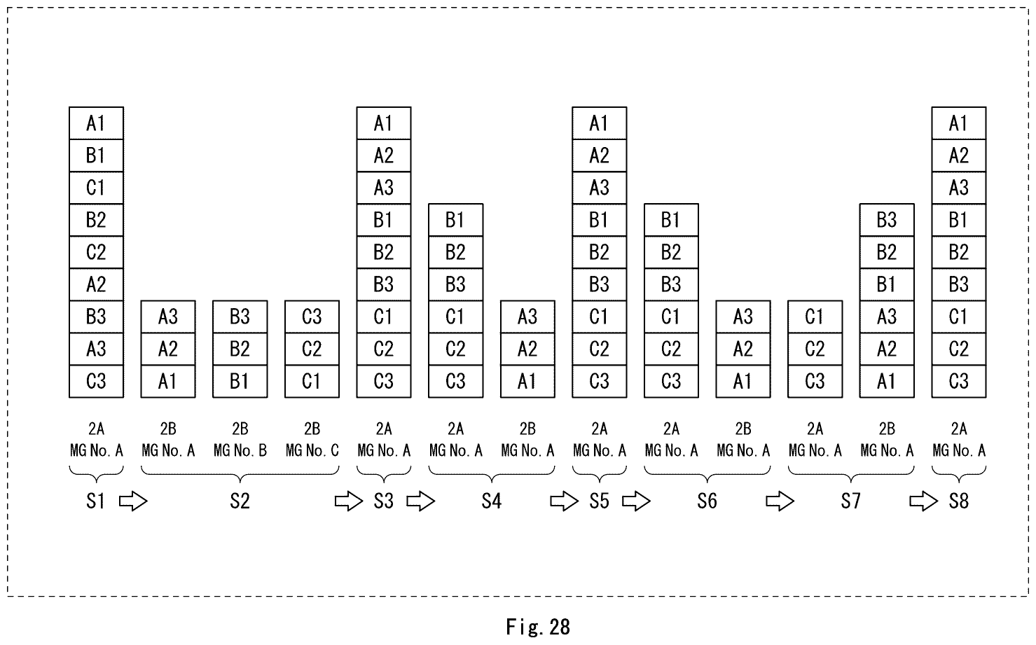

As illustrated in FIG. 24, a group-based sorting process means a process of sorting and re-arranging the Petri dishes S, . . . according to groups, from a state of a multi-stack of Petri dishes MS having randomly stacked Petri dishes S, . . . of each group. The sorting process is broadly divided into two types: sorting processes 1 to 3 which sort the Petri dishes S, . . . according to groups to form multi-stacks of Petri dishes MS according to groups; and sorting processes 4 and 5 which form one multi-stack of Petri dishes MS in a state in which the Petri dishes S, . . . were sorted according to groups. In the reading process of this sorting process, the control circuit 13 identifies the group to which the Petri dishes S of each stack of the multi-stack of Petri dishes MS belong, and stores the identification results to a recording medium of the control circuit 13.

### Group-based Sorting Process 1

Group-based Sorting Process 1 is illustrated in FIG. 25. Starting from Step 1, and the first-time measuring process is performed on all the Petri dishes S, . . . in Step 2. Alternatively, a colony counting process need not be performed but only an imaging process may be performed. Alternatively, the counting process and the imaging process need not be performed. Moreover, at this time, the reading process of the information recording medium assigned to the Petri dishes S is performed on all the Petri dishes S, . . . . After these processes, they are sorted at the second Petri dish stacking apparatus 2B, according to groups differentiated by means of the read results, to form multi-stacks of Petri dishes MS according to groups. In this case, the vertical order of the Petri dishes S of each group of the multi-stacks of Petri dishes MS becomes reversed with that of the original multi-stack of Petri dishes MS. Then, the re-stacking process is performed in Step 3.

Next, in Step 4, the second-time measuring process (if the measuring process was not performed in Step 2, the first-time measuring process) is performed on the Petri dishes S (A1 to A3) of one group (Group A). In this case, the transfer process and the measuring process are not performed on the Petri dishes S, . . . of the other groups (Group B and Group C). Then, when the measuring process of the last Petri dish S (A3) of one group (Group A) ends, the re-stacking process is performed in Step 5.

Thereafter, the measuring process is performed similarly on the Petri dishes S, . . . of any one group whose predetermined measuring time has arrived. Then, when the measuring process of the last Petri dish S of the relevant group ends, the re-stacking process is performed. The time interval of the measuring process is configured to be Group A<Group B<Group C.

As described above, in Group-based Sorting Process 1, Petri dishes S are sorted to form multi-stacks of Petri dishes MS according to groups. Therefore, compared to the aforementioned “Another Usage Example 1”, the measuring process can be performed continuously and at equal intervals on the Petri dishes S, . . . of the same group.

Moreover, if the measuring process was not performed in Step 2, the time interval of the measuring process of the Petri dishes S, . . . of the same group can be fixed from the first-time measuring process.

### Group-Based Sorting Process 2

Group-based Sorting Process 2 is illustrated in FIG. 26. Starting from Step 1, the reading process of the information recording medium assigned to the Petri dishes S is performed on all the Petri dishes S, . . . in Step 2. After the reading process, they are sorted according to groups differentiated by means of the read results to form multi-stacks of Petri dishes MS according to groups, in the magazine 22 of a plurality of magazine numbers (Magazine Numbers B, C, D) of the first Petri dish stacking apparatus 2A. Next, in Step 3, the first-time measuring process is performed on the Petri dishes S (A1 to A3) of one group (Group A). In this case, the transfer process and measuring process are not performed on the Petri dishes S, . . . of the other groups (Group B and Group C). After the measuring process, a multi-stack of Petri dishes MS of one group (Group A) is configured, in the magazine 22 of the original magazine number (Magazine Number A), in a state in which the vertical order of the Petri dishes S was reversed with that of the multi-stack of Petri dishes MS of one group (Group A) configured in the magazine 22 of a separate magazine number (Magazine Number B). Then, the re-stacking process is performed in Step 4.

Thereafter, the measuring process is performed similarly on the Petri dishes S, . . . of any one group whose predetermined measuring time has arrived. Then, when the measuring process of the last Petri dish S of the relevant group ends, the re-stacking process is performed. The time interval of the measuring process is configured to be Group A<Group B<Group C.

As described above, according to Group-based Sorting Process 2, after Step 2, the first-time measuring process can be performed without requiring the re-stacking process, and the time interval of the measuring process of the Petri dishes S, . . . of the same group can be fixed from the first-time measuring process.

### Group-based Sorting Process 3

Group-based Sorting Process 3 is illustrated in FIG. 27. Starting from Step 1, the first-time measuring process is performed on all the Petri dishes S, . . . in Step 2. Alternatively, a colony counting process need not be performed but only an imaging process may be performed. Alternatively, the counting process and the imaging process need not be performed. Moreover, at this time, the reading process of the information recording medium assigned to the Petri dishes S is performed on all the Petri dishes S, . . . . After these processes, they are sorted at the second Petri dish stacking apparatus 2B, according to groups differentiated by means of the read results, to form multi-stacks of Petri dishes MS according to groups. In this case, the vertical order of the Petri dishes S of each group of the multi-stacks of Petri dishes MS becomes reversed with that of the original multi-stack of Petri dishes MS. Therefore, the re-stacking process is performed in Step 3.

Next, in Step 4, the second-time measuring process (if the measuring process was not performed in Step 2, the first-time measuring process) is performed on the Petri dishes S, . . . of all groups. Then, when the measuring process of the last Petri dish S of all groups ends, the re-stacking process is performed in Step 5.

Thereafter, upon the arrival of a predetermined measuring time, the measuring process is similarly performed on the Petri dishes S, . . . of all groups. Then, when the measuring process of the last Petri dish S ends, the re-stacking process is performed. The time interval of the measuring process is configured to be Group A=Group B=Group C.

### Group-based Sorting Process 4

Group-based Sorting Process 4 is illustrated in FIG. 28. Starting from Step 1, the first-time measuring process is performed on all the Petri dishes S, . . . in Step 2. Alternatively, a colony counting process need not be performed but only an imaging process may be performed. Alternatively, both the counting process and the imaging process need not be performed. Moreover, at this time, the reading process of the information recording medium assigned to the Petri dishes S is performed on all the Petri dishes S, . . . . After these processes, they are sorted at the second Petri dish stacking apparatus 2B, according to groups differentiated by means of the read results, to form multi-stacks of Petri dishes MS according to groups. In this case, the vertical order of the Petri dishes S of each group of the multi-stacks of Petri dishes MS becomes reversed with that of the original multi-stack of Petri dishes MS. Therefore, the re-stacking process is performed in Step 3. The re-stacking process is performed so that the Petri dishes S, . . . according to groups are transferred from the second Petri dish stacking apparatus 2B to the first Petri dish stacking apparatus 2A, in the order reverse to that of the latter measuring process, and so that all the Petri dishes S, . . . are stacked in the same location to form one multi-stack of Petri dishes MS.

Next, in Step 4, the second-time measuring process (if the measuring process was not performed in Step 2, the first-time measuring process) is performed on the Petri dishes S (A1 to A3) of one group (Group A). In this case, the transfer process and the measuring process are not performed on the Petri dishes S, . . . of the other groups (Group B and Group C). Then, when the measuring process of the last Petri dish S (A3) of one group (Group A) ends, the re-stacking process is performed in Step 5.

Next, in Step 6, for the measuring process of the Petri dishes (B1 to B3) of a separate group (Group B), the Petri dishes S (A1 to A3) of one group (Group A) are transferred to the second Petri dish stacking apparatus 2B. Then, following this, in Step 7, the second-time measuring process (if the measuring process was not performed in Step 2, the first-time measuring process) is performed on the Petri dishes S (B1 to B3) of a separate group (Group B). In this case, the measuring process is not performed on the Petri dishes S, . . . of one group (Group A), and the transfer process and the measuring process are not performed on the Petri dishes S, . . . of the remaining group (Group C). Then, when the measuring process of the last Petri dish S (B3) of the separate group (Group B) ends, the re-stacking process is performed in Step 8.

Thereafter, the measuring process is performed similarly on the Petri dishes S, . . . of any one group whose predetermined measuring time has arrived. Then, when the measuring process of the last Petri dish S of the relevant group ends, the re-stacking process is performed. The time interval of the measuring process is configured to be Group A<Group B<Group C.

### Group-Based Sorting Process 5

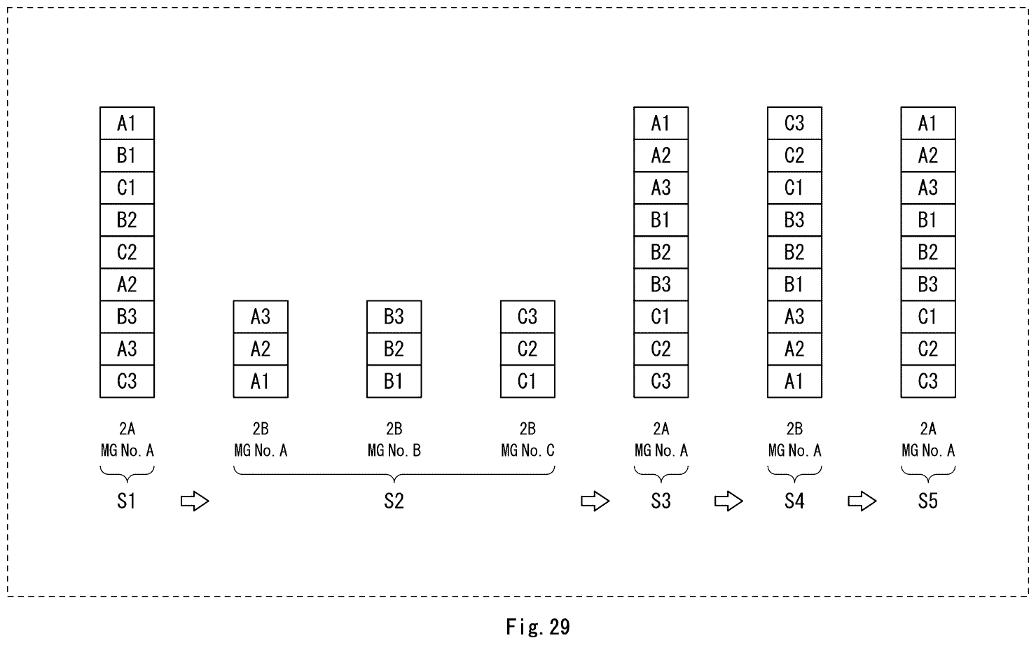

Group-based Sorting Process 5 is illustrated in FIG. 29. Starting from Step 1, the first-time measuring process is performed on all the Petri dishes S, . . . in Step 2. Alternatively, a colony counting process need not be performed but only an imaging process may be performed. Alternatively, both the counting process and the imaging process need not be performed. Moreover, at this time, the reading process of the information recording medium assigned to the Petri dishes S is performed on all the Petri dishes S, . . . . After these processes, these are sorted at the second Petri dish stacking apparatus 2B, according to groups differentiated by means of the read results, to form multi-stacks of Petri dishes MS according to groups. In this case, the vertical order of the Petri dishes S of the multi-stacks of Petri dishes MS of each group becomes reversed with that of the original multi-stack of Petri dishes MS. Therefore, the re-stacking process is performed in Step 3. The re-stacking process is performed so that the Petri dishes S, . . . according to groups are transferred from the second Petri dish stacking apparatus 2B to the first Petri dish stacking apparatus 2A, in the order reverse to that of the latter measuring process, and so that all the Petri dishes S, . . . are stacked in the same location to form one multi-stack of Petri dishes MS.

Next, in Step 4, the second-time measuring process (if the measuring process was not performed in Step 2, the first-time measuring process) is performed on the Petri dishes S, . . . of all groups. Then, when the measuring process of the last Petri dish S of all groups ends, the re-stacking process is performed in Step 5.

Thereafter, upon the arrival of a predetermined measuring time, the measuring process is similarly performed on the Petri dishes S, . . . of all groups. Then, when the measuring process of the last Petri dish S ends, the re-stacking process is performed. The time interval of the measuring process is configured to be Group A=Group B=Group C.

One example of an automatic measuring apparatus is provided with:
a placement section in which a multi-stack of containers can be placed, where
a sample has been put into each container and an information recording medium has been assigned to each container,
a container transferring apparatus which can transfer a container,
a measuring apparatus for measuring the sample in the container,
a reader for reading the information recording medium assigned to the container,
a control circuit for controlling the container transferring apparatus, the measuring apparatus and the reader, where
the control circuit
makes the container transferring apparatus take out a container from a top of the multi-stack of containers in the placement section in a manner of one container at a time and dispose the container sequentially on a top of another container in a predetermined location to form a multi-stack of containers in the predetermined location, or take out a container from a bottom of the multi-stack of containers in the placement section in a manner of one container at a time and dispose the container sequentially on a bottom of another container in a predetermined location to form a multi-stack of containers in the predetermined location,
makes the reader sequentially read information of the information recording medium assigned to the taken out container, and
makes the container transferring apparatus take out a container whose measurement timing has arrived, in accordance with the information of each information recording medium which was read, and transfer the container to the measuring apparatus.

When taking out the container from the top of the multi-stack of containers in the placement section, if a container whose measurement timing has not arrived is present on a container whose measurement timing has arrived, the control circuit performs control so that the container whose measurement timing has not arrived is taken out and transferred to a location separate to a location where the multi-stack of containers is present, without measuring the taken out container.

When taking out the container from the bottom of the multi-stack of containers in the placement section, if a container whose measurement timing has not arrived is present under a container whose measurement timing has arrived, the control circuit performs control so that the container whose measurement timing has not arrived is taken out and transferred to a location separate to a location where the multi-stack of containers is present, without measuring the taken out container.

### Another Usage Example 4 (Measurement Results-Based Sorting Process)

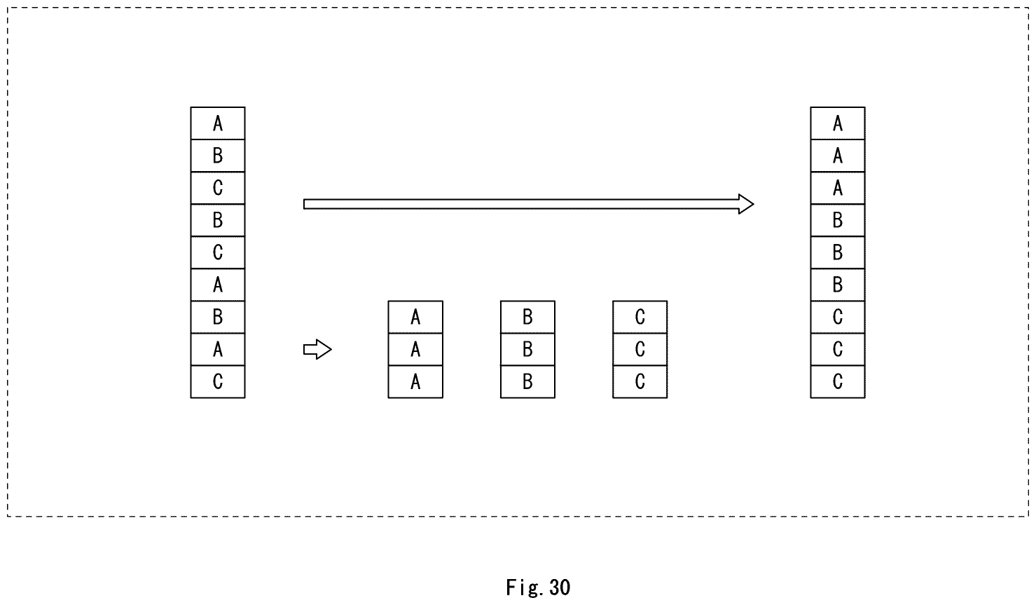

As illustrated in FIG. 30, a measured results-based sorting process means a process of categorizing measurement results into a plurality of groups according to predetermined conditions, and sorting and re-arranging the Petri dishes S, . . . according to groups. The sorting process is broadly divided into two types: a sorting process which sorts the Petri dishes S, . . . according to groups to form multi-stacks of Petri dishes MS according to groups; and a sorting process which forms one multi-stack of Petri dishes MS in a state in which the Petri dishes S, . . . are sorted according to groups.

### Measured Results-based Sorting Process 1

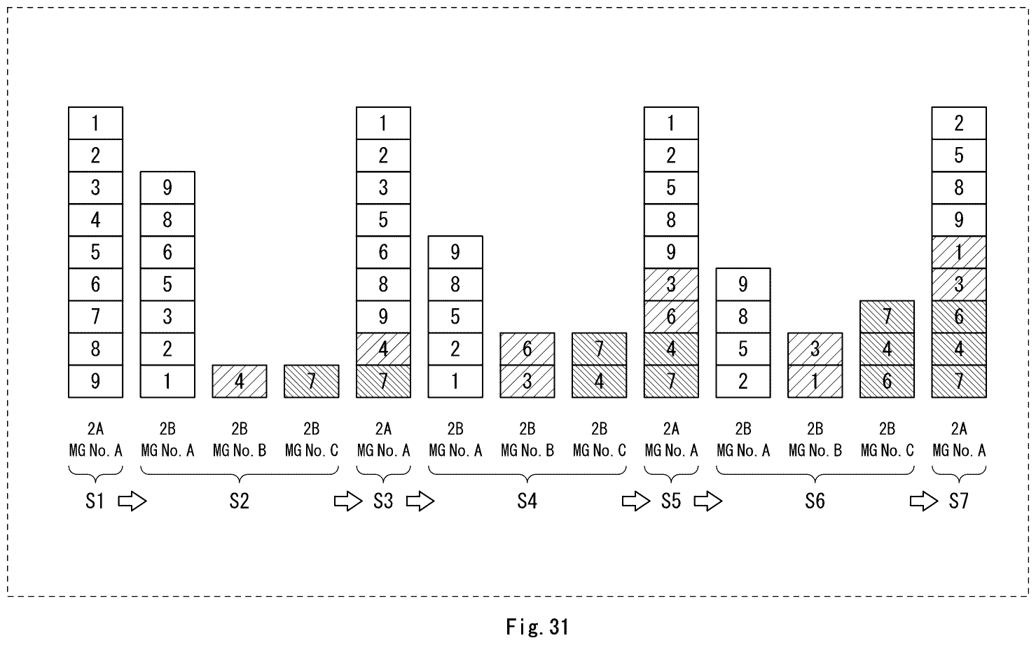

Measured Results-based Sorting Process 1 is illustrated in FIG. 31. Starting from Step 1, and the first-time measuring process is performed on all the Petri dishes S, . . . in Step 2. After the measuring process, they are sorted at the second Petri dish stacking apparatus 2B, according to groups based on measured results, to form multi-stacks of Petri dishes MS according to groups. Here, a group is categorized by the number of colonies counted by the measuring process (hereinafter in the same applies to each relevant usage example). The present example is divided into three Groups A to C, where those with 100 or fewer colonies are formed as Group A, those with more than 100 and less than 200 colonies are configured as Group B, and those with 200 or more colonies are configured as Group C. Then, at the second Petri dish stacking apparatus 2B: the Petri dishes S, . . . belonging to a group with the few colonies (Group A) are sorted in the magazine 22 of one magazine number (Magazine Number A); the Petri dishes S, . . . belonging to a group with more than the few colonies (Group B) are sorted in the magazine 22 of a separate magazine number (Magazine Number B), and the Petri dishes S, . . . belonging to a group with many colonies (Group C) are further sorted in the magazine 22 of a separate magazine number (Magazine Number C). In this case, the vertical order of the Petri dishes S of the multi-stacks of Petri dishes MS of each group becomes reversed with that of the original multi-stack of Petri dishes MS. Therefore, the re-stacking process is performed in Step 3. The re-stacking process is performed so that the Petri dishes S, . . . are transferred from the second Petri dish stacking apparatus 2B to the first Petri dish stacking apparatus 2A according to groups, in the order of groups with many colonies, and so that all the Petri dishes S, . . . are stacked in the same location to form one multi-stack of Petri dishes MS.

Next, in Step 4, when a predetermined measuring time arrives, the second-time measuring process is performed on the Petri dishes S, . . . of all groups. The groups to which each of the Petri dishes S belongs are determined again in accordance with the results of this measuring process. Then, when the measuring process of the last Petri dish S of all groups ends, the re-stacking process is performed in Step 5.

Next, in Step 6, when a predetermined measuring time further arrives, the third-time measuring process is performed on the Petri dishes S, . . . of all groups. Then, when the measuring process of the last Petri dish S of all groups ends, the re-stacking process is performed in Step 7.

Thereafter, upon the arrival of a predetermined measuring time, the measuring process is similarly performed on the Petri dishes S, . . . of all groups, and the groups to which each of the Petri dishes S belongs are determined in accordance with the results of the measuring process. Then, when the measuring process of the last Petri dish S ends, the re-stacking process is performed.

Thus, in Measured Results-based Sorting Process 1, the measuring process is continued even on the Petri dishes S, . . . of a group (Group C) whose number of colonies exceeded a threshold (in the present example: 200 colonies. Bad samples; hereinafter referred to as NG samples). Therefore, as in the samples whose number of colonies do not reach a threshold, captured images and measurement results can be continuously acquired and stored.

Moreover, in this case, due to the NG samples being disposed at the lower side of the multi-stack of Petri dishes MS, a person can immediately visually check the NG samples. This is also the same as the case in which the NG samples are disposed at the upper side of the multi-stack of Petri dishes MS.

The evaluation of the number of colonies and the evaluation of thresholds can be configured not only by using merely the total number of colonies as an object, but can also be configured by using, e.g., only the numbers of a specific microbe when a plurality of microbes can grow, as an object.

Moreover, samples with many colonies need not be configured as NG samples, but samples of which a fixed number of colonies is not attained after a fixed time may also be configured as NG samples.

### Measured Results-based Sorting Process 2

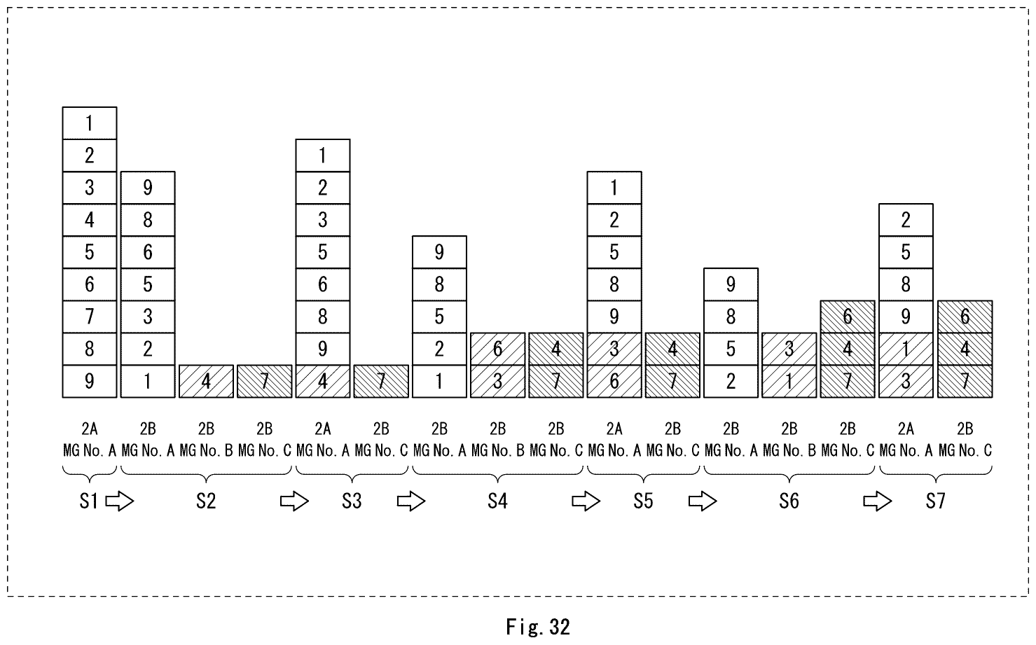

Measured Results-based Sorting Process 2 is illustrated in FIG. 32. Measured Results-based Sorting Process 2 differs from Measured Results-based Sorting Process 1 in that, in Measured Results-based Sorting Process 2, the Petri dishes S, . . . (NG samples) of the group whose number of colonies exceeded a threshold (Group C) are not subject to the re-stacking process and remain as-is in the sorted state. This can thereby eliminate wasteful processes such as the re-stacking process of the NG samples and the measuring process on the NG samples.

Moreover, in this case, a person can immediately visually check the NG samples because the NG samples are disposed separately from the multi-stack of Petri dishes MS where there are no NG samples.

### Measured Results-Based Sorting Process 3

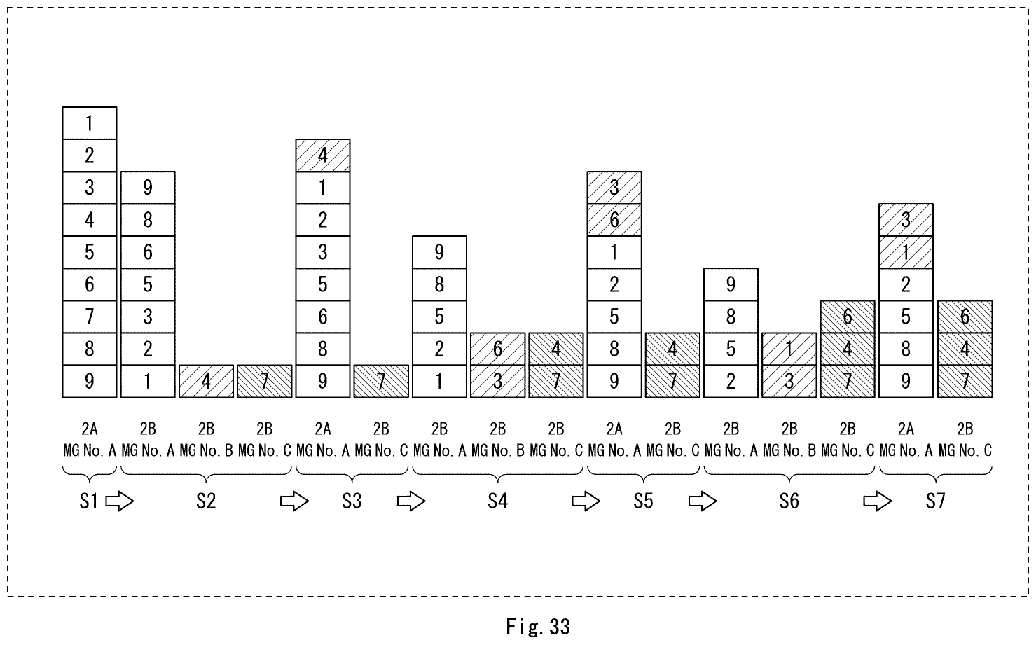

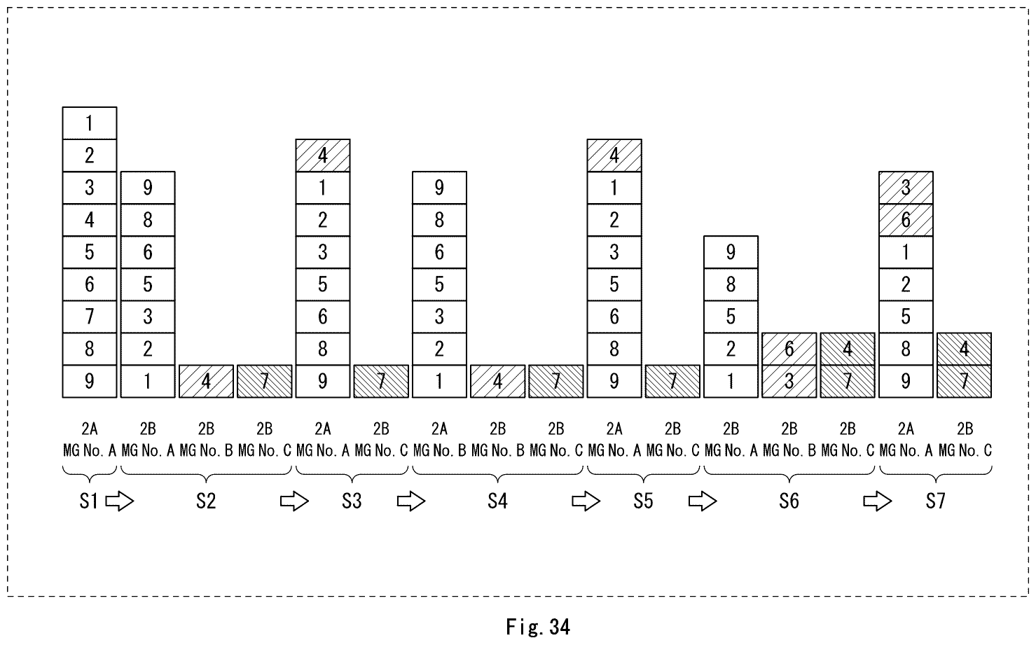

Measured Results-based Sorting Process 3 is illustrated in FIG. 33. Measured Results-based Sorting Process 3 differs from Measured Results-based Sorting Process 2 in that, in Measured Results-based Sorting Process 3, when the Petri dishes S, . . . of groups whose number of colonies do not reach a threshold (Group A and Group B) undergo the re-stacking process, the Petri dishes S, . . . of the group with a larger number of colonies (Group B) are stacked on the Petri dishes S, . . . of group with a smaller number of colonies (Group A), and are disposed at the upper side of the multi-stack of Petri dishes MS. Thereby, when visually checking group differences, a person can also check them immediately.

### Measured Results-Based Sorting Process 4

Measured Results-based Sorting Process 4 is illustrated in FIG. 34. Measured Results-based Sorting Process 4 has a basis on Measured Results-based Sorting Process 3, and of the groups whose numbers of colonies do not reach a threshold (Group A and Group B), the time interval of the measuring process is shortened (in the present example, by ½), and the number of times of the measuring process is increased (in the present example, doubles), on the Petri dishes S, . . . of a group with a larger number of colonies (Group B) which may be shifted to a group whose number of colonies exceeded a threshold (Group C).

Specifically, by the Nth measuring process (Step 2) and the N+1th measuring process (Step 6), the Petri dishes S, . . . of all groups whose numbers of colonies do not reach a threshold (Group A and Group B) are subject to the measuring process; however, a measuring process (Step 4) is additionally performed between these steps on the Petri dishes S, . . . of the group with a larger number of colonies (Group B) amongst them. Thereby, the Petri dishes S, . . . (NG samples) of a group whose number of colonies exceeded a threshold (Group C) can be found at an early stage.

One example of an automatic measuring apparatus is provided with:
a placement section in which a multi-stack of containers can be placed, where
a sample has been put into each container,
a container transferring apparatus which can transfer a container,
a measuring apparatus for measuring the sample in the containers,
a control circuit for controlling the container transferring apparatus and the measuring apparatus, where the control circuit
operates the container transferring apparatus so that a container is taken out from a top of the multi-stack of containers in the placement section in a manner of one container at a time, or a container is taken out from a bottom of the multi-stack of containers in the placement section in a manner of one container at a time, and the taken out container is transferred to the measuring apparatus,
makes the measuring apparatus measure the sample in the container transferred to the measuring apparatus, and
determines a group to which each container belongs in accordance with measured results of the measuring apparatus.

The control circuit may also make the container transferring apparatus sort and place the container according to groups.

The control circuit may also make the container transferring apparatus take out a container whose measurement timing has arrived in accordance with the group and transfer the container to the measuring apparatus.

When taking out the container from the top of the multi-stack of containers in the placement section, if a container whose measurement timing has not arrived is present on a container whose measurement timing has arrived, the control circuit performs control so that the container whose measurement timing has not arrived is taken out and transferred to a location separate to a location where the multi-stack of containers is present, without measuring the taken out container.

When taking out the container from the bottom of the multi-stack of containers in the placement section, if a container whose measurement timing has not arrived is present under a container whose measurement timing has arrived, the control circuit performs control so that the container whose measurement timing has not arrived is taken out and transferred to a location separate to a location where the multi-stack of containers is present, without measuring the taken out container.

The aforementioned “Rotation correction process in the measuring process”, “Equalization process of cultivation conditions”, “Another Usage Example 1”, “Another Usage Example 2”, “Another Usage Example 3” and “Another Usage Example 4” are achieved by means of control by the control circuit 13.

1. Container handling method including:
transferring a container from a placement section for placing a multi-stack of containers to a measuring apparatus for measuring a sample in the container, and then transferring the container from the measuring apparatus to a predetermined location, wherein the container is taken out from a top of the multi-stack of containers in the placement section in a manner of one container at a time and the container is disposed sequentially on a top of another container in the predetermined location to form a multi-stack of containers in the predetermined location, or wherein the container is taken out from a bottom of the multi-stack of containers in the placement section in a manner of one container at a time and the container is disposed sequentially on a bottom of another container in the predetermined location to form a multi-stack of containers in the predetermined location; and
at a predetermined timing after transferring a last container of the multi-stack of containers from the placement section to the measuring apparatus and before a subsequent measuring process on the multi-stack of containers, initially disposing the last container in a separately predetermined location, and thereafter, taking out a container from a top of the multi-stack of containers in the predetermined location in a manner of one container at a time and disposing the container sequentially on a top of the last container or another container in the separately predetermined location to form a multi-stack of containers in the separately predetermined location, or taking out a container from a bottom of the multi-stack of containers in the predetermined location in a manner of one container at a time and disposing the container sequentially on a bottom of the last container or another container in the separately predetermined location to form a multi-stack of containers in the separately predetermined location.

2. Automatic measuring apparatus, comprising:
a placement section in which a multi-stack of containers can be placed,
a measuring apparatus configured to measure a sample in a container,
a container transferring apparatus configured to transfer the container, and
a control circuit configured to control the container transferring apparatus, wherein
controlled by the control circuit, the container transferring apparatus:
transfers the container from the placement section to the measuring apparatus, and then from the measuring apparatus to a predetermined location, wherein the container is taken out from a top of the multi-stack of containers in the placement section in a manner of one container at a time and the container is disposed sequentially on a top of another container in the predetermined location to form a multi-stack of containers in the predetermined location, or wherein the container is taken out from a bottom of the multi-stack of containers in the placement section in a manner of one container at a time and the container is disposed sequentially on a bottom of another container in the predetermined location to form a multi-stack of containers in the predetermined location; and
at a predetermined timing after transferring a last container of the multi-stack of containers from the placement section to the measuring apparatus and before a subsequent measuring process on the multi-stack of containers, initially disposes the last container in a separately predetermined location, and thereafter, takes out a container from a top of the multi-stack of containers in the predetermined location in a manner of one container at a time and disposes the container sequentially on a top of the last container or another container in the separately predetermined location to form a multi-stack of containers in the separately predetermined location, or takes out a container from a bottom of the multi-stack of containers in the predetermined location in a manner of one container at a time and disposes the container sequentially on a bottom of the last container or another container in the separately predetermined location to form a multi-stack of containers in the separately predetermined location.

3. Automatic measuring apparatus according to claim 2, comprising
a temporary placement section in which a multi-stack of containers can be placed, wherein
the temporary placement section is set as the predetermined location, and
the placement section is set as the separately predetermined location.

4. Automatic measuring apparatus according to claim 3, wherein
the placement section and the temporary placement section are each a container stacking apparatus comprising
a rotary table, and
magazines installed at equally divided angular locations around a rotary center of the rotary table, and each enabled to accommodate a multi-stack of containers, so that the multi-stack of containers can be placed on the placement section and the temporary placement section.

5. Automatic measuring apparatus according to claim 3, wherein
the placement section and the temporary placement section are disposed in areas on both sides of the measuring apparatus.

6. Automatic measuring apparatus according to claim 3, comprising
an enclosure enclosing the placement section, the measuring apparatus, the temporary placement section and the container transferring apparatus.

7. Container handling method according to claim 1, wherein
a temporary placement section in which a multi-stack of containers can be placed, is set as the predetermined location and
the placement section is set as the separately predetermined location.

8. Container handling method according to claim 7, wherein
the placement section and the temporary placement section are disposed in areas on both sides of the measuring apparatus,
as an onward route, the container is transferred from the placement section to the measuring apparatus, and then transferred from the measuring apparatus to the temporary placement section, and
as a return route, the container is transferred from the temporary placement section to the placement section via the measuring apparatus.

9. Container handling method, including:
transferring a container from a placement section for placing a multi-stack of containers to a measuring apparatus for measuring a sample in the container, and then transferring the container from the measuring apparatus to a predetermined location, wherein the container is taken out from a top of the multi-stack of containers in the placement section in a manner of one container at a time and the container is disposed sequentially on a top of another container in the predetermined location to form a multi-stack of containers in the predetermined location, and
at a predetermined timing after transferring a last container of the multi-stack of containers from the placement section to the measuring apparatus and before a subsequent measuring process on the multi-stack of containers, initially disposing the last container in a separately predetermined location, and thereafter, taking out a container from a top of the multi-stack of containers in the predetermined location in a manner of one container at a time and disposing the container sequentially on a top of the last container or another container in the separately predetermined location to form a multi-stack of containers in the separately predetermined location.

10. Container handling method according to claim 9, wherein
a temporary placement section in which a multi-stack of containers can be placed, is set as the predetermined location and
the placement section is set as the separately predetermined location.

11. Container handling method according to claim 10, wherein
the placement section and the temporary placement section are disposed in areas on both sides of the measuring apparatus,
as an onward route, the container is transferred from the placement section to the measuring apparatus, and then transferred from the measuring apparatus to the temporary placement section, and
as a return route, the container is transferred from the temporary placement section to the placement section via the measuring apparatus.

12. Automatic measuring apparatus according to claim 4, wherein
the rotary table of the placement section and the rotary table of the temporary placement section rotate synchronously.

13. Automatic measuring apparatus according to claim 2, wherein
the container is a Petri dish, and
the measuring apparatus measures the number of colonies being cultivated in the Petri dish.
